SEQUENCE LISTING (48 sequences)

SEQ ID NO: 1 - RNA, 36 nt, Artificial Sequence
  SYNTHETIC RNA OLIGONUCLETIDE
cuuauauccaacacuucgugguuuuagagcuaugcu

SEQ ID NO: 2 - RNA, 36 nt, Artificial Sequence; identical to SEQ ID NO: 1
  SYNTHETIC RNA OLIGONUCLEOTIDE
  misc_feature (1)..(1): 5′-SpC3 spacer (propanediol)
  misc_feature (36)..(36): 3′-SpC3 spacer (propanediol)

SEQ ID NO: 3 - RNA, 36 nt, Artificial Sequence; identical to SEQ ID NO: 1
  SYNTHETIC RNA OLIGONUCLEOTIDE
  misc_feature (1)..(2): 2′-O-methyl moiety
  misc_feature (2)..(3): phosphorothioate internucleotoide linkage
  misc_feature (33)..(33): 2′-O-methyl moiety
  misc_feature (34)..(35): locked nucleic acid  internucleotoide linkage
  misc_feature (35)..(36): phosphorothioate internucleotoide linkage
  misc_feature (36)..(36): 2′-O-methyl moiety

SEQ ID NO: 4 - RNA, 36 nt, Artificial Sequence; identical to SEQ ID NO: 1
  SYNTHETIC RNA OLIGONUCLEOTIDE
  misc_feature (1)..(9): 2′-O-methyl moiety
  misc_feature (2)..(4): phosphorothioate internucleotide linkage
  misc_feature (20)..(20): 2′-O-methyl moiety
  misc_feature (24)..(36): 2′-O-methyl moiety
  misc_feature (34)..(36): phosphorothioate internucleotide linkage

SEQ ID NO: 5 - DNA, 36 bp, Artificial Sequence
  SYNTHETIC RNA-DNA SEQUENCE
  misc_feature (1)..(2): 2′-O-methyl moiety
  misc_feature (2)..(3): phosphorothioate internucleotide linkage
  misc_feature (33)..(35): locked nucleic acid internucleotide linkage
  misc_feature (35)..(36): phosphorothioate internucleotide linkage
  misc_feature (36)..(36): 2′-O-methyl moiety
cuuauauccaacacuucgugguuuuagagcuatgcu

SEQ ID NO: 6 - RNA, 36 nt, Artificial Sequence; identical to SEQ ID NO: 1
  SYNTHETIC RNA SEQUENCE
  misc_feature (1)..(2): 2′-O-methyl moiety
  misc_feature (2)..(3): phosphorothioate internucleotide linkage
  misc_feature (29)..(30): locked nucleic acid internucleotide linkage
  misc_feature (33)..(33): 2′-O-methyl moiety
  misc_feature (34)..(35): locked nucleic acid internucleotide linkage
  misc_feature (35)..(36): phosphorothioate internucleotide linkage
  misc_feature (36)..(36): 2′-O-methyl moiety

SEQ ID NO: 7 - RNA, 36 nt, Artificial Sequence; identical to SEQ ID NO: 1
  SYNTHETIC RNA OLIGONULCEOTIDE
  misc_feature (1)..(2): 2′-O-methyl moiety
  misc_feature (2)..(3): phosphorothioate internucleotide linkage
  misc_feature (29)..(30): locked nucleic acid internucleotide linkage
  misc_feature (31)..(33): 2′-O-methyl moiety
  misc_feature (34)..(35): locked nucleic acid internucleotide linkage
  misc_feature (35)..(36): phosphorothioate internucleotide linkage
  misc_feature (36)..(36): 2′-O-methyl moiety

SEQ ID NO: 8 - DNA, 36 bp, Artificial Sequence; identical to SEQ ID NO: 5
  SYNTHETIC RNA-DNA OLIGONUCLEOTIDE
  misc_feature (1)..(2): 2′-O-methyl
  misc_feature (2)..(3): phosphorothioate internucleotide linkage
  misc_feature (32)..(32): 2′-O-methyl
  misc_feature (33)..(35): locked nucleic acid internucleotide linkage
  misc_feature (35)..(36): phosphorothioate internucleotide linkage
  misc_feature (36)..(36): 2′-O-methyl

SEQ ID NO: 9 - RNA, 100 nt, Artificial Sequence
  SYNTHETIC RNA OLIGONUCLEOTIDE
  misc_feature (1)..(3): 2′-O-methyl moiety
  misc_feature (2)..(4): phosphorothioate internucleotide linkage
  misc_feature (97)..(99): 2′-O-methyl moiety
  misc_feature (98)..(100): phosphorothioate internucleotide linkage
cuuauauccaacacuucgugguuuuagagcuagaaauagcaaguuaaaauaaggcuaguccguuaucaacuugaaaaaguggcaccgagucggugcuuuu

SEQ ID NO: 10 - RNA, 36 nt, Artificial Sequence
  SYNTHETIC RNA OLIGONUCLEOTIDE
  misc_feature (1)..(1): 5′-SpC3 spacer (propanediol)
  misc_feature (36)..(36): 3′-SpC3 spacer (propanediol)
gaggcuauucugcccauuugguuuuagagcuaugcu

SEQ ID NO: 11 - RNA, 36 nt, Artificial Sequence; identical to SEQ ID NO: 10
  SYNTHETIC RNA SEQUENCE
  misc_feature (1)..(2): 2′-O-methyl moiety
  misc_feature (2)..(3): phosphorothioate internucleotide linkage
  misc_feature (33)..(33): 2′-O-methyl moiety
  misc_feature (34)..(35): locked nucleic acid internucleotide linkage
  misc_feature (35)..(36): phosphorothioate internucleotide linkage
  misc_feature (36)..(36): 2′-O-methyl moiety

SEQ ID NO: 12 - DNA, 36 bp, Artificial Sequence
  SYNTHETIC RNA-DNA OLIGONUCLEOTIDE
  misc_feature (1)..(2): 2′-O-methyl moiety
  misc_feature (2)..(3): phosphorothioate internucleotide linkage
  misc_feature (32)..(32): 2′-O-methyl moiety
  misc_feature (33)..(35): locked nucleic acid internucleotide linkage
  misc_feature (35)..(36): phosphorothioate internucleotide linkage
  misc_feature (36)..(36): 2′-O-methyl moiety
gaggcuauucugcccauuugguuuuagagcuatgcu

SEQ ID NO: 13 - RNA, 100 nt, Artificial Sequence
  SYNTHETIC RNA OLIGONUCLEOTIDE
  misc_feature (1)..(2): 2′-O-methyl moiety
  misc_feature (2)..(3): phosphorothioate internucleotide linkage
  misc_feature (97)..(99): 2′-O-methyl moiety
  misc_feature (98)..(100): phosphorothioate internucleotide linkage
gaggcuauucugcccauuugguuuuagagcuagaaauagcaaguuaaaauaaggcuaguccguuaucaacuugaaaaaguggcaccgagucggugcuuuu

SEQ ID NO: 14 - RNA, 36 nt, Artificial Sequence
  SYNTHETIC RNA OLIGONUCLEOTIDE
  misc_feature (1)..(1): 5′-SpC3 (propanediol)
  misc_feature (36)..(36): 3′-SpC3 (propanediol)
uggcacugagcucccagaucguuuuagagcuaugcu

SEQ ID NO: 15 - RNA, 36 nt, Artificial Sequence; identical to SEQ ID NO: 14
  SYNTHETIC RNA OLIGONUCLEOTIDE
  misc_feature (1)..(2): 2′-O-methyl moiety
  misc_feature (2)..(3): phosphorothioate internucleotide linkage
  misc_feature (33)..(33): 2′-O-methyl moiety
  misc_feature (34)..(35): locked nucleic acid internucleotide linkage
  misc_feature (35)..(36): phosphorothioate internucleotide linkage
  misc_feature (36)..(36): 2′-O-methyl moiety

SEQ ID NO: 16 - DNA, 36 bp, Artificial Sequence
  SYNTHETIC RNA-DNA OLIGONUCLEOTIDE
  misc_feature (1)..(2): 2′-O-methyl moiety
  misc_feature (2)..(3): phosphorothioate internucleotide linkage
  misc_feature (32)..(32): 2′-O-methyl moiety
  misc_feature (33)..(35): locked nucleic acid internucleotide linkage
  misc_feature (35)..(36): phosphorothioate internucleotide linkage
  misc_feature (36)..(36): 2′-O-methyl moiety
uggcacugagcucccagaucguuuuagagcuatgcu

SEQ ID NO: 17 - RNA, 100 nt, Artificial Sequence
  SYNTHETIC RNA OLIGONUCLEOTIDE
  misc_feature (1)..(2): 2′-O-methyl moiety
  misc_feature (2)..(3): phosphorothioate internucleotide linkage
  misc_feature (97)..(99): 2′-O-methyl moiety
  misc_feature (98)..(100): phosphorothioate internucleotide linkage
uggcacugagcucccagaucguuuuagagcuagaaauagcaaguuaaaauaaggcuaguccguuaucaacuugaaaaaguggcaccgagucggugcuuuu

SEQ ID NO: 18 - RNA, 36 nt, Artificial Sequence
  SYNTHETIC RNA OLIGONUCLEOTIDE
  misc_feature (1)..(1): 5′-SpC3 (propanediol)
  misc_feature (36)..(36): 3′-SpC3 (propanediol)
aggacaaguucucugaguucguuuuagagcuaugcu

SEQ ID NO: 19 - RNA, 36 nt, Artificial Sequence; identical to SEQ ID NO: 18
  SYNTHETIC RNA OLIGONUCLEOTIDE
  misc_feature (1)..(2): 2′-O-methyl moiety
  misc_feature (2)..(3): phosphorothioate internucleotide linkage
  misc_feature (33)..(33): 2′-O-methyl moiety
  misc_feature (34)..(35): locked nucleic acid internucleotide linkage
  misc_feature (35)..(36): phosphorothioate internucleotide linkage
  misc_feature (36)..(36): 2′-O-methyl moiety

SEQ ID NO: 20 - DNA, 35 bp, Artificial Sequence
  SYNTHETIC RNA-DNA OLIGONUCLEOTIDE
  misc_feature (1)..(2): 2′-O-methyl moiety
  misc_feature (2)..(3): phosphorothioate internucleotide linkage
  misc_feature (31)..(31): 2′-O-methyl moiety
  misc_feature (32)..(34): locked nucleic acid internucleotide linkage
  misc_feature (34)..(35): phosphorothioate internucleotide linkage
  misc_feature (35)..(35): 2′-O-methyl moiety
aggacaaguucucugaguucguuuuagagcatgcu

SEQ ID NO: 21 - RNA, 100 nt, Artificial Sequence
  SYNTHETIC RNA OLIGONUCLEOTIDE
  misc_feature (1)..(2): 2′-O-methyl moiety
  misc_feature (2)..(3): phosphorothioate internucleotide linkage
  misc_feature (97)..(99): 2′-O-methyl moiety
  misc_feature (98)..(100): phosphorothioate internucleotide linkage
aggacaaguucucugaguucguuuuagagcuagaaauagcaaguuaaaauaaggcuaguccguuaucaacuugaaaaaguggcaccgagucggugcuuuu

SEQ ID NO: 22 - RNA, 36 nt, Artificial Sequence
  SYNTHETIC RNA OLIGONUCLEOTIDE
  misc_feature (1)..(1): 5′-SpC3 spacer (propanediol)
  misc_feature (36)..(36): 3′-SpC3 spacer (propanediol)
ccccuccaaccuggaauuccguuuuagagcuaugcu

SEQ ID NO: 23 - RNA, 36 nt, Artificial Sequence; identical to SEQ ID NO: 22
  SYNTHETIC RNA OLIGONUCLEOTIDE
  misc_feature (1)..(2): 2′-O-methyl moiety
  misc_feature (2)..(3): phosphorothioate internucleotide linkage
  misc_feature (33)..(33): 2′-O-methyl moiety
  misc_feature (34)..(35): locked nucleic acid internucleotide linkage
  misc_feature (35)..(36): phosphorothioate internucleotide linkage
  misc_feature (36)..(36): 2′-O-methyl moiety

SEQ ID NO: 24 - DNA, 36 bp, Artificial Sequence
  SYNTHETIC RNA-DNA OLIGONUCLEOTIDE
  misc_feature (1)..(2): 2′-O-methyl moiety
  misc_feature (2)..(3): phosphorthioate internucleotide linkage
  misc_feature (32)..(32): 2′-O-methyl moiety
  misc_feature (33)..(35): locked nucleic acid internucleotide linkage
  misc_feature (35)..(36): phosphorthioate internucleotide linkage
  misc_feature (36)..(36): 2′-O-methyl moiety
ccccuccaaccuggaauuccguuuuagagcuatgcu

SEQ ID NO: 25 - RNA, 100 nt, Artificial Sequence
  SYNTHETIC RNA OLIGONUCLEOTIDE
  misc_feature (1)..(2): 2′-O-methyl moiety
  misc_feature (2)..(3): phosphorothioate internucleotide linkage
  misc_feature (97)..(99): 2′-O-methyl moiety
  misc_feature (98)..(100): phosphorothioate internucleotide linkage
ccccuccaaccuggaauuccguuuuagagcuagaaauagcaaguuaaaauaaggcuaguccguuaucaacuugaaaaaguggcaccgagucggugcuuuu

SEQ ID NO: 26 - RNA, 36 nt, Artificial Sequence
  SYNTHETIC RNA OLIGONUCLEOTIDE
  misc_feature (1)..(1): 5′-SpC3 spacer (propanediol)
  misc_feature (36)..(36): 3′-SpC3 spacer (propanediol)
gcugcuguagcugauuccauguuuuagagcuaugcu

SEQ ID NO: 27 - RNA, 36 nt, Artificial Sequence; identical to SEQ ID NO: 26
  SYNTHETIC RNA OLIGONUCLEOTIDE
  misc_feature (1)..(2): 2′-O-methyl moiety
  misc_feature (2)..(3): phosphorothioate internucleotide linkage
  misc_feature (33)..(33): 2′-O-methyl moiety
  misc_feature (34)..(35): locked nucleic acid internucleotide linkage
  misc_feature (35)..(36): phosphorothioate internucleotide linkage
  misc_feature (36)..(36): 2′-O-methyl moiety

SEQ ID NO: 28 - DNA, 36 bp, Artificial Sequence
  SYNTHETIC RNA-DNA OLIGONUCLEOTIDE
  misc_feature (1)..(2): 2′-O-methyl moiety
  misc_feature (2)..(3): phosphorothioate internucleotide linkage
  misc_feature (32)..(32): 2′-O-methyl moiety
  misc_feature (33)..(35): locked nucleic acid internucleotide linkage
  misc_feature (35)..(36): phosphorothioate internucleotide linkage
  misc_feature (36)..(36): 2′-O-methyl moiety
gcugcuguagcugauuccauguuuuagagcuatgcu

SEQ ID NO: 29 - RNA, 100 nt, Artificial Sequence
  SYNTHETIC RNA OIGONUCLEOTIDE
  misc_feature (1)..(2): 2′-O-methyl moiety
  misc_feature (2)..(3): phosphorothioate internucleotide linkage
  misc_feature (97)..(99): 2′-O-methyl moiety
  misc_feature (98)..(100): phosphorothioate internucleotide linkage
gcugcuguagcugauuccauguuuuagagcuagaaauagcaaguuaaaauaaggcuaguccguuaucaacuugaaaaaguggcaccgagucggugcuuuu

SEQ ID NO: 30 - RNA, 36 nt, Artificial Sequence; identical to SEQ ID NO: 1
  SYNTHETIC RNA OLIGONUCLEOTIDE
  misc_feature (1)..(1): 5′-SpC3 spacer (propanediol)
  misc_feature (1)..(1)
  misc_feature (36)..(36): 3′-SpC3 spacer (propanediol)

SEQ ID NO: 31 - RNA, 36 nt, Artificial Sequence; identical to SEQ ID NO: 1
  SYNTHETIC RNA OLIGONUCLEOTIDE
  misc_feature (1)..(3): 2′-O-methyl moiety
  misc_feature (2)..(4): phosphorothioate internucleotide linkage
  misc_feature (34)..(36): 2′-O-methyl moiety
  misc_feature (34)..(36): phosphorothioate internucleotide linkage

SEQ ID NO: 32 - RNA, 36 nt, Artificial Sequence; identical to SEQ ID NO: 1
  SYNTHETIC RNA OLIGONUCLEOTIDE
  misc_feature (1)..(9): 2′-O-methyl moiety
  misc_feature (2)..(4): phosphorothioate internucleotide linkage
  misc_feature (24)..(36): 2′-O-methyl moiety
  misc_feature (34)..(36): phosphorothioate internucleotide linkage

SEQ ID NO: 33 - RNA, 67 nt, Artificial Sequence
  SYNTHETIC RNA OLIGONUCLEOTIDE
  misc_feature (1)..(8): 2′-O-methyl moiety
  misc_feature (2)..(3): phosphorothioate internucleotide linkage
  misc_feature (38)..(67): 2′-O-methyl moiety
  misc_feature (66)..(67): phosphorothioate internucleotide linkage
agcauagcaaguuaaaauaaggcuaguccguuaucaacuugaaaaaguggcaccgagucggugcuuu

SEQ ID NO: 34 - RNA, 67 nt, Artificial Sequence; identical to SEQ ID NO: 33
  SYNTHETIC RNA OLIGONUCLEOTIDE

SEQ ID NO: 35 - protein, 1368 aa, Artificial Sequence [sequence omitted]

SEQ ID NO: 36 - RNA, 42 nt, Artificial Sequence
  SYNTHETIC RNA OLIGONUCLEOTIDE
cuuauauccaacacuucgugguuuuagagcuaugcuguuuug

SEQ ID NO: 37 - RNA, 36 nt, Artificial Sequence; identical to SEQ ID NO: 1
  SYNTHETIC RNA OLIGONUCLEOTIDE

SEQ ID NO: 38 - RNA, 89 nt, Artificial Sequence
  SYNTHETIC RNA OLIGONUCLEOTIDE
guuggaaccauucaaaacagcauagcaaguuaaaauaaggcuaguccguuaucaacuugaaaaaguggcaccgagucggugcuuuuuuu

SEQ ID NO: 39 - RNA, 99 nt, Artificial Sequence
  SYNTHETIC RNA OLIGONUCLEOTIDE
cuuauauccaacacuucgugguuuuagagcuagaaauagcaaguuaaaauaaggcuaguccguuaucaacuugaaaaaguggcaccgagucggugcuuu

SEQ ID NO: 40 - RNA, 36 nt, Artificial Sequence; identical to SEQ ID NO: 1
  SYNTHETIC RNA OLIGONUCLEOTIDE
  misc_feature (1)..(14): 2′-O-methyl moiety
  misc_feature (2)..(4): phosphorothioate internucleotide linkage
  misc_feature (17)..(18): 2′-O-methyl moiety
  misc_feature (20)..(21): 2′-O-methyl moiety
  misc_feature (26)..(36): 2′-O-methyl moiety
  misc_feature (34)..(36): phosphorothioate internucleotide linkage

SEQ ID NO: 41 - RNA, 16 nt, Artificial Sequence
  SYNTHETIC RNA OLIGONUCLEOTIDE
  misc_feature (13)..(13): 2′-O-methyl moiety
  misc_feature (14)..(16): locked nucleic acid  internucleotide linkage
  misc_feature (15)..(15): phosphorothioate internucleotide linkage
  misc_feature (16)..(16): 2′-O-methyl moiety
guuuuagagcuaugcu

SEQ ID NO: 42 - DNA, 17 bp, Artificial Sequence
  SYNTHETIC RNA-DNA OLIGONUCLEOTIDE
  misc_feature (13)..(13): 2′-O-methyl moiety
  misc_feature (14)..(16): locked nucleic acid internucleotide linkage
  misc_feature (16)..(17): phosphorothioate internucleotide linkage
  misc_feature (17)..(17): 2′-O-methyl moiety
guuuuagagcuuatgcu

SEQ ID NO: 43 - RNA, 16 nt, Artificial Sequence
  SYNTHETIC RNA OLIGONUCLEOTIDE
  misc_feature (9)..(10): locked nucleic acid internucleotide linkage
  misc_feature (13)..(13): 2′-O-methyl moiety
  misc_feature (14)..(15): locked nucleic acid internucleotide linkage
  misc_feature (15)..(16): phosphorothioate internucleotide linkage
  misc_feature (16)..(16): 2′-O-methyl moiety
guuuuagagcuaugcu

SEQ ID NO: 44 - RNA, 16 nt, Artificial Sequence
  SYNTHETIC RNA OLIGONUCLEOTIDE
  misc_feature (9)..(10): locked nucleic acid internucleotide linkage
  misc_feature (11)..(13): 2′-O-methyl moiety
  misc_feature (14)..(15): locked nucleic acid internucleotide linkage
  misc_feature (15)..(16): phosphorothioate internucleotide linkage
  misc_feature (16)..(16): 2′-O-methyl moiety
guuuuagagcuaugcu

SEQ ID NO: 45 - RNA, 36 nt, Artificial Sequence
  SYNTHETIC RNA OLIGONUCLEOTIDE
ggugagugagugugugcgugguuuuagagcuaugcu

SEQ ID NO: 46 - RNA, 36 nt, Artificial Sequence; identical to SEQ ID NO: 45
  SYNTHETIC RNA OLIGONUCLEOTIDE
  misc_feature (1)..(1): 5′-SpC3 Spacer (propanediol)
  misc_feature (36)..(36): 3′-SpC3 Spacer (propanediol)

SEQ ID NO: 47 - RNA, 36 nt, Artificial Sequence; identical to SEQ ID NO: 45
  SYNTHETIC RNA SEQUENCE
  misc_feature (2)..(7): phosphorothioate internucleotide linkage
  misc_feature (31)..(36): phosphorothioate internucleotide linkage

SEQ ID NO: 48 - DNA, 8068 bp, Artificial Sequence
  SYNTHETIC DNA OLIGONUCLEOTIDE
ggggaattgtgagcggataacaattcccctgtagaaataattttgtttaactttaataaggagatataccatggacaaaaagtactctattggcctggatatcgggaccaacagcgtcgggtgggctgttatcaccgacgagtataaagtaccttcgaaaaagttcaaagtgctgggcaacaccgatcgccattcaatcaaaaagaacttgattggtgcgctgttgtttgactccggggaaaccgccgaggcgactcgccttaaacgtacagcacgtcgccggtacactcggcgtaagaatcgcatttgctatttgcaggaaatctttagcaacgagatggcaaaagtcgatgactcgtttttccaccgcctcgaggaaagctttctggtggaggaagacaaaaagcatgagcgtcacccgatcttcggcaacattgtcgatgaagtagcgtatcatgaaaaatacccaaccatttaccacttacgcaaaaagctggtggacagcactgacaaagctgatttgcgccttatctatttagccctggcacatatgattaagtttcgtggtcacttcctgatcgaaggagacttaaatcccgacaacagtgatgttgataaattgtttattcagcttgtccaaacttacaatcaactgttcgaggaaaacccgatcaatgcctccggtgtggatgcaaaagccattttaagtgcacgccttagcaagtcccgtcgcttagaaaaccttatcgcgcagctgcccggcgagaaaaagaatggtttgtttgggaaccttattgccttgagcttaggcctcaccccgaatttcaaaagtaatttcgatcttgcagaagacgccaaattacaactgtcgaaggatacttatgatgacgatctcgataatctgttagcgcagattggtgaccaatacgccgatctttttctggcggctaaaaatctgagcgacgccatcttgctttcggatattctccgcgttaacaccgaaatcacgaaagcgcctcttagtgccagcatgattaaacgttatgatgaacaccaccaggacctgaccttactcaaagcgttggttcgccagcaactgccagagaagtacaaagaaatcttctttgatcagtcaaagaatggttatgccggctatattgacgggggtgcaagccaagaggaattctacaaatttatcaagcctattctggagaaaatggatggcaccgaagagttattggtgaagcttaaccgtgaagacctcctgcggaaacagcgcacattcgataatggttcgatcccacaccaaatccatttgggggagttacacgctattttgcgtcgccaggaagacttttaccctttcctgaaggataaccgggagaaaattgagaagatccttacctttcgtattccgtattacgtaggccccttagcacggggtaatagccgtttcgcgtggatgacacggaagtcggaagagacgatcaccccgtggaacttcgaagaggtagtcgacaagggcgcatcagcgcagtcttttattgaacgtatgacgaatttcgataaaaacttgcccaatgagaaggtgcttccgaaacattccttgttatatgaatattttacagtttacaacgagctgaccaaggttaaatacgtgacggaaggaatgcgcaagcccgcttttcttagcggtgagcaaaaaaaggcgatcgtcgacctgttattcaaaacgaatcgtaaggtgactgtaaagcaactcaaagaagattacttcaaaaagattgagtgcttcgacagcgtcgaaatctctggggtagaggatcggtttaacgcaagtttaggtacctaccatgacctgcttaaaatcattaaggataaagacttcttagataatgaagagaacgaagatattctcgaggacatcgtcttgacgttaaccttatttgaggatcgtgaaatgattgaggaacgcctcaaaacttatgcccacctgttcgacgataaggtgatgaagcagctgaaacgtcggcgctacacaggatggggccgcttgagtcgcaaacttattaacggaatccgtgacaagcaatccggcaaaacgattctggatttcttgaagtcggacggatttgctaatcgcaacttcatgcagttgatccatgatgactccctgacttttaaagaggatattcaaaaggcgcaggttagtggtcaaggcgacagcttacacgaacacatcgcaaatttggctggttcgccggccattaaaaaggggatcctccagaccgtgaaagttgtagatgagcttgttaaggtcatgggtcgtcataagcccgaaaacatcgtgattgaaatggcgcgggagaatcaaacgacccagaaaggacaaaagaatagccgtgaacggatgaagcggatcgaggaaggcattaaagagctggggtctcaaatcttgaaggaacaccctgtggagaacactcagctccaaaatgaaaaactttacctgtactatttgcagaacggacgcgatatgtacgtggaccaagagttggatattaatcggctgagtgactacgacgttgatcatatcgtcccgcagagcttcctcaaagacgattctattgacaataaggtactgacgcgctctgataaaaaccgtggtaagtcggacaacgtgccctccgaagaggttgtgaaaaagatgaaaaattattggcgccagcttttaaacgcgaagctgatcacacaacgtaaattcgataatttgaccaaggctgaacggggtggcctgagcgagttagataaggcaggatttattaaacgccagttagtggagactcgtcaaatcaccaaacatgtcgcgcagattttggacagccggatgaacaccaagtacgatgaaaatgacaaactgatccgtgaggtgaaagtcattactctgaagtccaaattagttagtgatttccggaaggactttcaattctacaaagtccgtgaaattaataactatcatcacgcacatgacgcgtacctgaatgcagtggttgggaccgcccttatcaagaaatatcctaagctggagtcggagtttgtctatggcgactataaggtatacgatgttcgcaaaatgattgcgaaatctgagcaggagatcggtaaggcaaccgcaaaatatttcttttactcaaacattatgaatttctttaagacagaaatcactctggccaacggggagattcgcaaacgtccgttgatcgaaacaaacggcgagactggcgaaattgtttgggacaaagggcgtgatttcgcgacggtgcgcaaggtactgagcatgcctcaagtcaatattgttaagaaaaccgaagtgcagacgggcgggttttccaaggaaagcatcttacccaaacgtaattcagataaacttattgcacgcaaaaaggactgggatccgaaaaagtatggaggcttcgacagtccaaccgtagcctactctgttctcgttgtagcgaaagtagaaaagggtaaatccaagaaactgaaatctgtcaaggagttgcttggaatcaccattatggagcgtagctccttcgagaagaacccgattgactttctggaagccaaaggatataaagaggtcaagaaagatcttatcattaagctgcctaagtattcactcttcgagctggaaaatggtcgtaaacgcatgctcgcttctgccggcgagttgcagaagggcaatgaattagcacttccatcaaagtacgttaacttcctgtatttggccagccattacgagaaactgaaggggtctccagaggacaacgaacagaaacaattatttgtagagcagcacaagcattatcttgatgaaatcattgagcaaatttccgaattcagtaaacgcgtaatcctggccgatgcaaacctcgacaaggtgctgagcgcttacaataagcatcgcgacaaacctatccgtgagcaggctgaaaatatcattcacctgttcacattaacgaacctgggcgctccggccgcttttaaatatttcgacacgacaatcgaccgtaagcgctataccagtacgaaagaagtgttggatgcgacccttattcaccagtcaattacaggattatatgagacccgtatcgaccttagccaattaggtggggattaagagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

(19) United States
(12) Patent Application Publication
(10) Pub. No.: US 2018/0179523 A1
(43) Pub. Date: Jun. 28, 2018

(54) CRISPR-BASED COMPOSITIONS AND METHODS OF USE

(71) Applicant: INTEGRATED DNA TECHNOLOGIES, INC., CORALVILLE, IA (US)

(72) Inventors: Michael Allen Collingwood, North Liberty, IA (US); Ashley Mae Jacobi, Tiffin, IA (US); Garrett Richard Rettig, Coralville, IA (US); Mollie Sue Schubert, Cedar Rapids, IA (US); Mark Aaron Behlke, Coralville, IA (US); Christopher Anthony Vakulskas, North Liberty, IA (US); Rolf Turk, Iowa City, IA (US)

(21) Appl. No.: 15/881,684

(22) Filed: Jan. 26, 2018

Related U.S. Application Data
(63) Continuation-in-part of application No. 15/299,590, filed on Oct. 21, 2016.
Application No. 15/299,590 is a division of application No. 14/975,709, filed on Dec. 18, 2015, now Pat. No. 9,840,702.
(60) Provisional application No. 62/239,546, filed on Oct. 9, 2015.
(60) Provisional application No. 62/093,588, filed on Dec. 18, 2014.

(51) Int. Cl. C12N 15/11

(52) U.S. Cl. CPC C12N 15/111; C12N 2310/10; C12N 2310/322; C12N 2320/51; C12N 2310/346; C12N 2310/3231; C12N 2310/3533; C12N 2310/321; C12N 2310/317; C12N 2310/315; C12N 2310/20; C12N 2310/531; C12N 2310/3521; C12N 2320/53

(57) ABSTRACT

This invention pertains to modified compositions for use in CRISPR systems, and their methods of use. In particular, length-modified and chemically-modified forms of crRNA are described for use as a reconstituted guide RNA for interaction with Cas9 of CRISPR systems. The resultant length-modified and chemically-modified forms of crRNA are economical to produce and can be tailored to have unique properties relevant to their biochemical and biological activity in the context of the CRIPSR Cas9 endonuclease system.

### CROSS-REFERENCE TO RELATED APPLICATIONS

This application is a continuation-in-part of U.S. patent application Ser. No. 15/299,590, filed Oct. 21, 2016, entitled “CRISPR-BASED COMPOSITIONS AND METHODS OF USE,” which is a divisional of U.S. patent application Ser. No. 14/975,709, filed Dec. 18, 2015, entitled “CRISPR-BASED COMPOSITIONS AND METHODS OF USE,” now U.S. Pat. No. 9,840,702, published Dec. 12, 2017, which claims benefit of priority under 35 U.S.C. 119 to U.S. provisional patent applications bearing Ser. Nos. 62/093,588 and 62/239,546, filed Dec. 18, 2014 and Oct. 9, 2015, and entitled “CRISPR-BASED COMPOSITIONS AND METHODS OF USE,” the contents of which are herein incorporated by reference in their entirety.

### SEQUENCE LISTING

The instant application contains a Sequence Listing that has been submitted in ASCII format via EFS-Web and is hereby incorporated by reference in its entirety. The ASCII copy, created on DATE, is named XXXX.txt, and is XXX,XXX bytes in size.

### FIELD OF THE INVENTION

This invention pertains to modified compositions for use in CRISPR systems, and their methods of use.

### BACKGROUND OF THE INVENTION

The use of clustered regularly interspaced short palindromic repeats (CRISPR) and associated Cas proteins (CRISPR-Cas system) for site specific DNA cleavage has shown great potential for a number of biological applications. CRISPR is used for genome editing; the genome-scale-specific targeting of transcriptional repressors (CRISPRi) and activators (CRISPRa) to endogenous genes; and other applications of RNA-directed DNA targeting with Cas enzymes.

CRISPR-Cas systems are native to bacteria and Archaea to provide adaptive immunity against viruses and plasmids. There are three classes of CRISPR-Cas systems that could potentially be adapted for research and therapeutic reagents, but Type-II CRISPR systems have a desirable characteristic in utilizing a single CRISPR associated (Cas) nuclease (specifically Cas9) in a complex with the appropriate guide RNAs—either a 2-part RNA system similar to the natural complex in bacteria comprising a CRISPR-activating RNA:trans-activating crRNA (crRNA:tracrRNA) pair or an artificial chimeric single-guide-RNA (sgRNA)—to mediate double-stranded cleavage of target DNA. In mammalian systems, these RNAs have been introduced by electroporation as well as transfection of DNA cassettes containing RNA Pol III promoters (such as U6 or H1) driving RNA transcription, viral vectors, and single-stranded RNA following in vitro transcription (see Xu, T. et al., Appl Environ Microbiol, 2014. 80(5):1544-52).

In the CRISPR-Cas9 system, using, for example, the system present in Streptococcus pyogenes as an example (S. py. or Spy), native crRNAs are about 42 nucleotides long, containing a 5′-region of about 20 bases complementary to a target sequence (also referred to as a protospacer sequence) and a 3′ region typically about 22 bases long that corresponds to a complementary region of the tracrRNA sequence. The native tracrRNAs are about 85-90 bases long, having a 5′-region complementary to the crRNA as well as about 10 noncomplementary bases upstream this region. The remaining 3′ region of the tracrRNA includes secondary structures (herein referred to as the “tracrRNA 3′-tail”).

Jinek et al. extensively investigated the portions of the crRNA and tracrRNA that are required for proper functioning of the CRISPR-Cas9 system (Science, 2012. 337(6096): p. 816-21). They devised a truncated crRNA:tracrRNA fragment that could still function in CRISPR-Cas9 wherein the crRNA was the wild type 42 nucleotides and the tracrRNA was truncated to 75 nucleotides. They also developed an embodiment wherein the crRNA and tracrRNA are attached with a linker loop, forming a single guide RNA (sgRNA), which varies between 99-123 nucleotides in different embodiments. The configuration of the native 2-part crRNA:tracrRNA complex is shown in FIG. 1 and the 99 nucleotide embodiment of the artificial sgRNA single guide is shown in FIG. 2.

At least two groups have elucidated the crystal structure of Streptococcus pyogenes Cas9 (SpyCas9). In Jinek, M. et al., the structure did not show the nuclease in complex with either a guide RNA or target DNA. They carried out molecular modeling experiments to reveal predictive interactions between the protein in complex with RNA and DNA (Science, 2014. 343, p. 1215, DOI: 10.1126/science/1247997).

In Nishimasu, H. et al., the crystal structure of SpyCas9 is shown in complex with sgRNA and its target DNA at 2.5 angstrom resolution (Cell, 2014. 156(5): p. 935-49, incorporated herein in its entirety). The crystal structure identified two lobes to the Cas9 enzyme: a recognition lobe (REC) and a nuclease lobe (NUC). The sgRNA:target DNA heteroduplex (negatively charged) sits in the positively charged groove between the two lobes. The REC lobe, which shows no structural similarity with known proteins and therefore likely a Cas9-specific functional domain, interacts with the portions of the crRNA and tracrRNA that are complementary to each other.

Another group, Briner et al. (Mol Cell, 2014. 56(2): p. 333-9, incorporated herein in its entirety), identified and characterized the six conserved modules within native crRNA:tracrRNA duplexes and sgRNA.

The CRISPR-Cas9 system is utilized in genomic engineering as follows: a portion of the crRNA hybridizes to a target sequence, a portion of the tracrRNA hybridizes to a portion of the crRNA, and the Cas9 nuclease binds to the entire construct and directs cleavage. The Cas9 contains two domains homologous to endonucleases HNH and RuvC, wherein the HNH domain cleaves the DNA strand complementary to the crRNA and the RuvC-like domain cleaves the noncomplementary strand. This results in a blunt double-stranded break in the genomic DNA 3 base pairs upstream the PAM site. When repaired by non-homologous end joining (NHEJ) the break is typically shifted by 1 or more bases, leading to disruption of the natural DNA sequence and in many cases leading to a frameshift mutation if the event occurs in the coding exon of a protein-encoding gene. The break may also be repaired by homology dependent recombination (HDR), which permits insertion of new genetic material via experimental manipulation into the cut site created by Cas9 cleavage.

Some of the current methods for guide RNA delivery into mammalian cells include transfection of double-stranded DNA (dsDNA) containing RNA Pol III promoters for endogenous transcription, viral delivery, transfection of RNAs as in vitro transcription (IVT) products, or microinjection of IVT products. There are disadvantages to each of these methods. Unmodified exogenous RNA introduced into mammalian cells is known to initiate the innate immune response via recognition by Toll-like Receptors (TLRs), RIG-I, OASI and others receptors that recognize pathogen-associated molecular patterns (PAMPs). However, in most published studies, RNA which has been in vitro transcribed (IVT) by a T7 RNA polymerase is delivered to the cells. This type of RNA payload has been shown to be a trigger for the innate immune response. The alternative delivery methods described above each have their own disadvantages as well. For example, dsDNA cassettes can lead to integration, guide RNA transcription driven endogenously by a RNA Pol II promoter can persist constitutively, and the amount of RNA transcribed is uncontrollable.

RNA is quickly degraded by nucleases present in serum and in cells. Unmodified CRISPR RNA triggers (crRNAs, tracrRNAs, and sgRNAs) made by IVT methods or chemical synthesis are quickly degraded during delivery or after delivery to mammalian cells. Greater activity would be realized if the RNA was chemically modified to gain nuclease resistance. The most potent degradative activity present in serum and in cells is a 3′-exonuclease (Eder et al., Antisense Research and Development 1:141-151, 1991). Thus “end blocking” a synthetic oligonucleotide often improves nuclease stability. Chemical modification of single-stranded antisense oligonucleotides (ASOs) and double-stranded small interfering RNAs (siRNAs) has been well studied and successful approaches are in practice today (for reviews, see: Kurreck, Eur. J. Biochem., 270:1628-1644, 2003; Behlke, Oligonucleotides, 18:305-320, 2008; Lennox et al., Gene Therapy, 18:1111-1120, 2011). It is therefore desirable to devise chemical modification strategies for use with the RNA components of CRISPR/Cas.

Additional chemical modifications strategies rely on the use of Locked Nucleic Acids (LNA). Locked nucleic acids are modified and contain a bridge group between the 2′ oxygen and the 4′ carbon of the ribose moiety. LNA modified oligonucleotides have been show to enhance thermostability of duplexed RNA, DNA, or RNA/DNA hybrids. Additionally it has been shown that LNA modified oligonucleotides can increase the nuclease resistance of the oligonucleotide (for reviews, see: Kurreck, Nucleic Acids Res., 30, 1911-1918, 2002; Crinelli, Nucleic Acids Res., 30, 2435-2443, 2002).

While the basic toolbox of chemical modifications available is well known to those with skill in the art, the effects that site-specific modification have on the interaction of a RNA species and an effector protein are not easily predicted and effective modification patterns usually must be empirically determined. In some cases, sequence of the RNA may influence the effectiveness of a modification pattern, requiring adjustment of the modification pattern employed for different sequence contexts, making practical application of such methods more challenging.

There is therefore a need to modify the guide RNA to reduce its toxicity to cells and to extend the lifespan and functionality in mammalian cells while still performing their intended purpose in the CRISPR-Cas system. Addition of chemical modifications can also allow the gRNA to be functional at a lower dosage, as well as increase activity for lower performing gRNA sites while maintaining similar indel profiles. The methods and compositions of the invention described herein provide RNA and modified RNA oligonucleotides for use in a CRISPR-Cas system. These and other advantages of the invention, as well as additional inventive features, will be apparent from the description of the invention provided herein.

### BRIEF SUMMARY OF THE INVENTION

This invention pertains to modified compositions for use in CRISPR systems, and their methods of use. The compositions include modified internucleotide linkages and 2′-O-alkyl and 2′-O-fluoro modified RNA oligonucleotides to serve as the guides strands (crRNA:tracrRNA or sgRNA) for the CRISPR-Cas system. Furthermore, compositions included modified nucleotides and LNA or BNA modified RNA oligonucleotides. Compositions also include end-modifications such as an inverted-dT base or other non-nucleotide modifiers that impeded exonuclease attack (such as the propanediol group (C3 spacer), napthyl-azo modifier, or others as are well known in the art).

In a first aspect, an isolated crRNA comprising a length-modified and chemically modified form of formula (I) is provided:

5′-X—Z-3′ (I).

X is a target-specific protospacer domain and Z is a tracrRNA-binding domain. The tracrRNA binding domain further comprises at least one chemically modified nucleotide. The isolated crRNA is active in a Clustered Regularly Interspaced Short Palindromic Repeats (CRISPR)/CRISPR-associated protein endonuclease system.

In a second aspect, a method of performing gene editing is provided. The method includes a step of contacting a candidate editing target site locus with an active CRISPR/Cas endonuclease system having a suitable crRNA. The crRNA has a tracrRNA binding domain. The tracrRNA binding domain further comprises at least one chemically modified nucleotide.

In a third aspect, a method of performing gene editing is provided. The method includes the step of contacting a candidate editing target site locus in bacteria with an active CRISPR/Cas endonuclease system having a suitable crRNA. The crRNA has a tracrRNA binding domain. The tracrRNA binding domain further comprises at least one chemically modified nucleotide.

### BRIEF DESCRIPTION OF THE DRAWINGS

FIG. 1 is an illustration of a wild-type (WT) natural 2-part crRNA:tracrRNA complex with a 42 base unmodified crRNA (SEQ ID No. 36) and an 89 base unmodified tracrRNA (SEQ ID No. 38). Lowercase letters represent RNA.

FIG. 2 is an illustration of a 99 base artificial single-guide RNA (SEQ ID NO: 39 (sgRNA) that fuses the crRNA and tracrRNA elements into a single sequence through the addition of a new hairpin loop. Lowercase letters represent RNA.

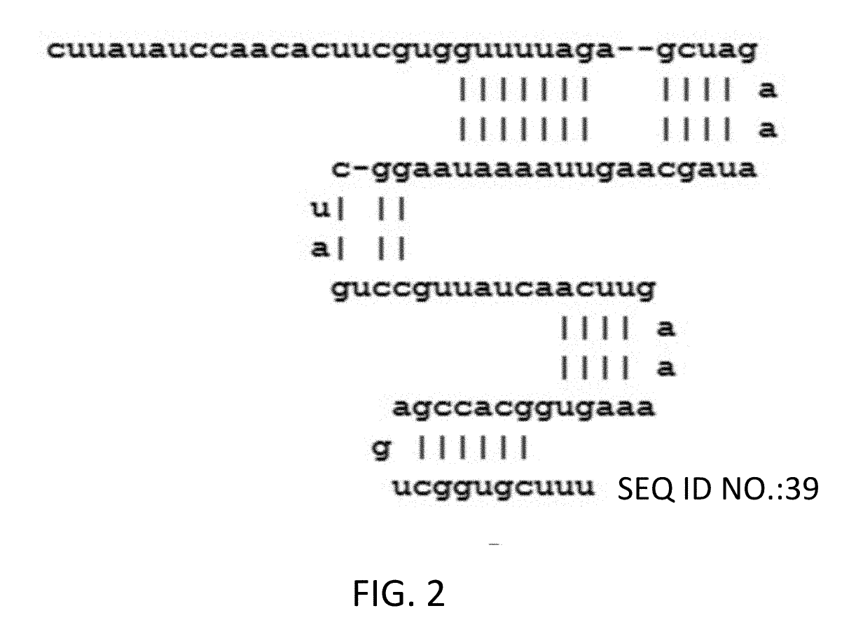

FIG. 3 is an illustration of a truncated 2-part crRNA:tracrRNA complex with a 36 base crRNA (SEQ ID No. 37) and a 67 base tracrRNA (SEQ ID No. 34). Lowercase letters represent RNA.

FIG. 4 is a schematic showing structure of one embodiment of an optimized truncated and chemically-modified crRNA (SEQ ID No. 40). Length is 36 bases. RNA is lower case and 2′OMe RNA is uppercase. Phosphorothioate (PS) internucleotide linkages are indicated by “*”. Residues which lead to substantial loss of function when converted from RNA to 2′OMe RNA are identified by large arrows and residues which lead to a moderate loss of function when converted from RNA to 2′OMe RNA are identified by small arrows. The 5′-end 20 base protospacer target-specific guide domain is indicated, which in this case is sequence specific to the human HPRT1 gene. The 3′-end 16 base tracrRNA binding domain is indicated.

FIG. 5 is a plot showing the functional gene editing observed using the T7E1 assay in HEK293 cells in a dose dependent manner using unmodified and truncated crRNA:tracrRNA (duplexed SEQ ID No. 37 and SEQ ID No. 34), modified and duplexed crRNA:tracrRNA (duplexed SEQ ID No. 2 and SEQ ID No. 33), LNA modified crRNA (SEQ ID No. 3) duplexed with modified tracrRNA (SEQ ID No. 33), and heavily modified crRNA (SEQ ID No. 4) duplexed with modified tracrRNA (SEQ ID No. 33).

FIG. 6. is a plot showing the dose dependent functional gene editing observed using the T7E1 assay in HEK293 cells using modified crRNA (SEQ ID No. 2) duplexed with modified tracrRNA (SEQ ID No. 33), and LNA modified crRNA mod1 (SEQ ID No. 3) duplexed with tracrRNA (SEQ ID No. 33), LNA modified crRNA mod2 (SEQ ID No. 5) duplexed with tracrRNA (SEQ ID No. 33), LNA modified crRNA mod 3 (SEQ ID No. 6) duplexed with tracrRNA (SEQ ID No. 33), LNA modified crRNA mod4 (SEQ ID No. 7) duplexed with tracrRNA (SEQ ID No. 33).

FIG. 7. is a plot showing the functional gene editing observed using the T7E1 assay in HEK293 cells using modified crRNA, LNA modified crRNAs, and modified sgRNA targeting different genomic regions at 2 doses.

FIG. 8. is a plot showing the functional gene editing observed using the T7E1 assay in Jurkat cells using unmodified two part crRNA/tracrRNA duplex (duplexed SEQ ID No. 1 and SEQ ID No. 34), minimally modified crRNA (SEQ ID No. 30) duplexed with modified tracrRNA (SEQ ID No. 33), medium modified crRNA (SEQ ID No. 31) duplexed with modified tracrRNA (SEQ ID No. 33), and heavy mod crRNA (SEQ ID No. 32) duplexed with modified tracrRNA (SEQ ID No. 33) delivered with Cas9 mRNA.

FIG. 9. is a plot showing the functional gene editing observed using the T7E1 assay in Jurkat cells. The plot compares medium modified crRNA (SEQ ID No. 31) duplexed with modified tracrRNA (SEQ ID No. 33), LNA mod1 crRNA (SEQ ID No. 3) duplexed with modified tracrRNA (SEQ ID No. 33), LNA mod2 crRNA (SEQ ID No. 4) duplexed with modified tracrRNA (SEQ ID No. 33).

### DETAILED DESCRIPTION OF THE INVENTION

Aspects of this invention relate to modified compositions for use in CRISPR systems, and their methods of use.

The term “oligonucleotide,” as used herein, refer to polydeoxyribonucleotides (containing 2-deoxy-D-ribose), polyribonucleotides (containing D-ribose), and to any other type of polynucleotide which is an N glycoside of a purine or pyrimidine base (a single nucleotide is also referred to as a “base” or “residue”). There is no intended distinction in length between the terms “nucleic acid”, “oligonucleotide” and “polynucleotide”, and these terms can be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs. An oligonucleotide may comprise ribonucleotides, deoxyribonucleotides, modified nucleotides (e.g., nucleotides with 2′ modifications, synthetic base analogs, etc.) or combinations thereof.

Compositions of the present invention include any modification that potentially reduces activation of the innate immune system. Modifications can be placed or substituted at a conventional phosphodiester linkage, at the ribose sugar, or at the nucleobase of RNA. Such compositions could include, for example, a modified nucleotide such as 2′-O-methly-modified RNAs. Further compositions could include, for example, a modified nucleotide such as LNA modified RNAs. Additional compositions could include a modified nucleotide containing one or more 2′O-methyl modifications and/or LNA modified nucleotides.

More broadly, the term “modified nucleotide” refers to a nucleotide that has one or more modifications to the nucleoside, the nucleobase, pentose ring, or phosphate group. For example, modified nucleotides exclude ribonucleotides containing adenosine monophosphate, guanosine monophosphate, uridine monophosphate, and cytidine monophosphate and deoxyribonucleotides containing deoxyadenosine monophosphate, deoxyguanosine monophosphate, deoxythymidine monophosphate, and deoxycytidine monophosphate. Modifications include those naturally occurring that result from modification by enzymes that modify nucleotides, such as methyltransferases. Modified nucleotides also include synthetic or non-naturally occurring nucleotides. Modifications also include base analogs and universal bases. Synthetic or non-naturally occurring modifications in nucleotides include those with 2′ modifications, e.g., 2′-O-alkyl (including 2′-O-methyl), 2′-fluoro, 2′-methoxyethoxy, 2′-allyl, 2′-O-[2-(methylamino)-2-oxoethyl], 4′-thio, bicyclic nucleic acids, 4′-CH2-O-2′-bridge, 4′-(CH2)2-O-2′-bridge, 2′-LNA, and 2′-O—(N-methylcarbamate) or those comprising base analogs. Such modified groups are described, e.g., in Eckstein et al., U.S. Pat. No. 5,672,695, Matulic-Adamic et al., U.S. Pat. No. 6,248,878, Wengel et al., U.S. Pat. No. 6,670,461, and Imanishi et al., U.S. Pat. No. 6,268,490.

The use of 2′-O-methyl has been documented in siRNA literature (See Behlke, M. A., Oligonucleotides, 2008. 18(4): p. 305-19) as well as in mRNA delivery (see Sahin, U. et al., Nat Rev Drug Discov, 2014. 13(10): p. 759-80). Sahin et al., describes modifications of mRNA therapeutics that extend beyond 2′-OMe modification and “non-immunogenic” mRNA.

The use of LNAs to protect oligonucleotides from nuclease degradation has been documented in literature. A fully modified LNA sequence has been reported to be fully resistant towards the 3′-exonuclease SVPDE (Frieden et al., 2003) whereas only minor protection against the same enzyme is obtained with one LNA monomer in the 3′-end or in the middle of a sequence. End blocked sequences, i.e. LNA-DNA-LNA gapmers, display a high stability in human serum compared to similar 2′-OMe modified sequences (Kurreck et al., 2002). Another study showed that two terminal LNA monomers provided protection against a Bal-31 exonucleolytic degradation (Crinelli et al., 2002). LNA oligonucleotides can be delivered into cells using standard cationic transfection, electroporation, or microinjection.

The term “ribonucleotide” encompasses natural and synthetic, unmodified and modified ribonucleotides. Modifications include changes to the sugar moiety, to the base moiety and/or to the linkages between ribonucleotides in the oligonucleotide.

The term “Cas9 protein” encompasses wild-type and mutant forms of Cas9 having biochemical and biological activity when combined with a suitable guide RNA (for example sgRNA or dual crRNA:tracrRNA compositions) to form an active CRISPR-Cas endonuclease system. This includes orthologs and Cas9 variants having different amino acid sequences from the Streptococcus pyogenese Cas9 employed as example in the present invention.

The term “length-modified,” as that term modifies RNA, refers to a shortened or truncated form of a reference RNA lacking nucleotide sequences or an elongated form of a reference RNA including additional nucleotide sequences.

The term “chemically-modified,” as that term modifies RNA, refers to a form of a reference RNA containing a chemically-modified nucleotide or a non-nucleotide chemical group covalently linked to the RNA. Chemically-modified RNA, as described herein, generally refers to synthetic RNA prepared using oligonucleotide synthesis procedures wherein modified nucleotides are incorporated during synthesis of an RNA oligonucleotide. However, chemically-modified RNA also includes synthetic RNA oligonucleotides modified with suitable modifying agents post-synthesis.

It will be understood by one of skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction components which includes the oligonucleotides of the invention.

Applicants have discovered novel crRNA oligonucleotide compositions that display robust and increased activity in the Clustered Regularly Interspaced Short Palindromic Repeats (CRISPR)-CRISPR associated (Cas) (CRISPR-Cas) endonuclease system. The oligonucleotide compositions include length-modified forms of crRNA as well as chemically-modified forms of crRNA. The length-modified forms of crRNA enable one to prepare active forms of these RNAs with cost-effective and efficient oligonucleotide synthesis protocols routinely available. The chemically-modified forms of crRNA provide one with active agents tunable with certain specific properties, such as improved stability in cellular and in vivo contexts. The length-modified forms of crRNA can also include modifications, thereby enabling access to a broad range of compositions having activity in CRISPR-Cas endonuclease system contexts. These oligonucleotide compositions and their properties in the CRISPR-Cas endonuclease system are described below.

Length-Modified Forms of crRNA

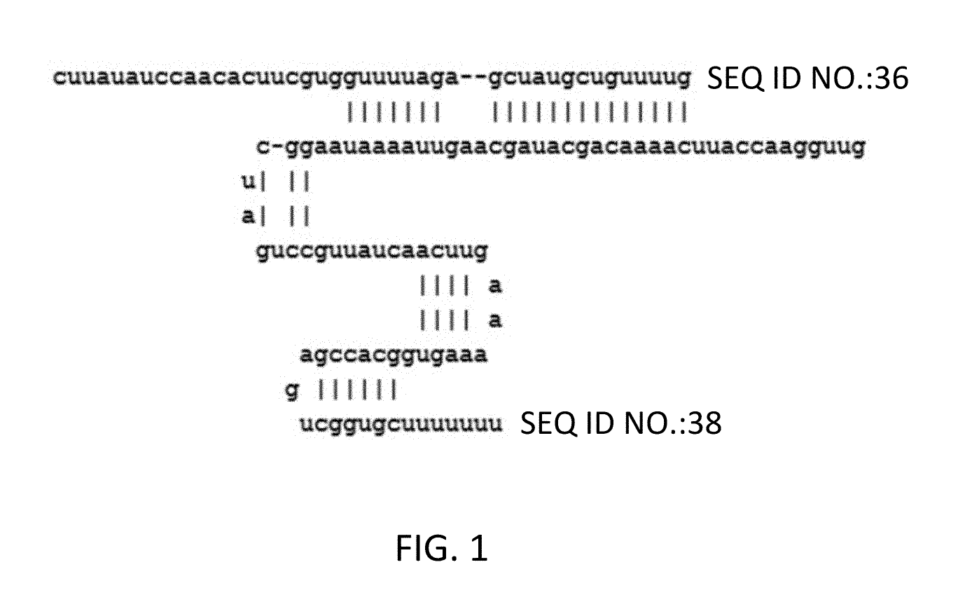

FIG. 1 depicts a representation of the wild-type S. pyogenes crRNA:tracrRNA complex, wherein an exemplary isolated crRNA (SEQ ID No. 36) is paired with an isolated tracrRNA (SEQ ID No. 38). In a first aspect, an isolated tracrRNA including a length modified form of SEQ ID NO. 38 is provided. The isolated tracrRNA displays activity in the CRISPR-Cas endonuclease system. In one respect, the isolated tracrRNA includes a length-modified form of SEQ ID NO. 38 nucleotide having deleted sequence information. In some embodiments, the length-modified form of SEQ ID NO. 38 includes shortened or truncated forms of SEQ ID NO. 38, wherein SEQ ID NO. 38 can be shortened by 1 to 20 nucleotides at the 5′-end and by 1-10 nucleotides at the 3′-end. Such shortened or truncated forms of SEQ ID NO. 38 retain activity when paired with a functionally competent crRNA in the CRISPR-Cas endonuclease system. Where shortening of the 5′-end of the tracrRNA is performed and extends into sequence that pairs with the 3′-end of the crRNA, improved activity may be obtained using chemical modifications that enhance binding affinity in these domains. Where shortening of the 3′-end of the crRNA is performed and extends into sequence that pairs with the 5′-end of the tracrRNA, improved activity may be obtained using chemical modifications that enhance binding affinity in these domains. Preferred examples of a length-modified form of SEQ ID No. 38 having a shortened or truncated form include SEQ ID No. 33 or SEQ ID No. 34. For each of the foregoing exemplary length-modified forms of SEQ ID No. 38 having a shortened or truncated form can consist of chemically non-modified nucleotides.

In a second aspect, an isolated crRNA comprising a length-modified form of formula (I) is provided:

5′-X—Z-3′ (I),

wherein X represents sequences including a target-specific protospacer domain, and Z represents sequences including a tracrRNA-binding domain.

The target-specific protospacer domain (X domain of formula (I)) typically includes about twenty nucleotides having complementarity to a region of DNA targeted by the CRISPR-Cas endonuclease system. The tracrRNA-binding domain (the Z domain of formula (I)) typically includes about 20 nucleotides in most CRISPR endonuclease systems (in the native S.py. version, this domain is 22 nucleotides). The isolated crRNA displays activity in the CRISPR-Cas endonuclease system.

In one respect, the isolated crRNA includes a length modified form of formula (I) having deleted sequence information. In some embodiments, the length-modified form of formula (I) includes shortened or truncated forms of formula (I), wherein formula (I) can be shortened by 1-8 nucleotides at the 3′-end of the Z domain. The length-modified form of formula (I) can be shortened at the 5-end of the X-domain to accommodate a target-specific protospacer domain having 17, 18, 19 or 20 nucleotides. Highly preferred examples of such length-modified form of formula (I) include target-specific protospacer domain having 19 or 20 nucleotides. The exemplary length-modified forms of formula (I) having a shortened or truncated form with a target-specific protospacer (X-domain) of 17-20 nucleotides in length and/or lacking 1-8 nucleotides at the 3′-end of the Z-domain can consist of chemically non-modified nucleotides.

Such shortened or truncated forms of formula (I) retain activity when paired with a competent tracrRNA in the CRISPR-Cas endonuclease system. Preferred embodiments of isolated crRNA of formula (I) having a length modified form of formula (I) can include chemically non-modified nucleotides and chemically modified nucleotides.

Chemically-Modified Forms of crRNA

In a third aspect, an isolated crRNA including a chemically-modified nucleotide is provided. The isolated crRNA displays activity in the CRISPR-Cas endonuclease system.

In one respect, the isolated crRNA includes a chemically-modified nucleotide having a modification selected from a group consisting of a ribose modification, an end-modifying group, and internucleotide modifying linkage. Exemplary ribose modifications include 2′O-alkyl (e.g., 2′OMe), 2′F, and bicyclic nucleic acid (including locked nucleic acid (LNA)). Exemplary end-modifying groups include a propanediol (C3) spacer and napthyl-azo modifier (N,N-diethyl-4-(4-nitronaphthalen-1-ylazo)-phenylamine, or “ZEN”), and an inverted-dT residue. Exemplary internucleotide modifying linkages include phosphorothioate modification. In one respect, the isolated crRNA having a chemically-modified form include crRNA of formula (I) and length-modified forms thereof. Preferred shortened or truncated forms of crRNA of formula (I) having a chemically-modified nucleotide include SEQ ID NOs.:2-7. These particular isolated crRNA species represent “universal” crRNAs having a chemically-modified nucleotide motif showing high activity when combined with a competent tracrRNA in the CRISPR-Cas endonuclease system. Yet other examples of isolated chemically-modified crRNA with robust activity in the CRISPR-Cas endonuclease system are presented in the Examples.

In another respect different variants of chemically modified crRNA are provided including variants optimized for performance in mammalian cells and variants optimized for performance in bacteria.

The foregoing isolated, length-modified and chemically-modified crRNA preferably include chemical modifications at the 2′-OH groups (for example, 2′OMe, 2′F, bicyclic nucleic acid, locked nucleic acid, among others) and end-blocking modifications (for example, ZEN, C3 spacer, inverted-dT). Use of both types of general modifications provides isolated, length-modified and chemically-modified of crRNA with biochemical stability and immunologic tolerance for isolated, length-modified and chemically-modified of crRNA in biological contexts.

The foregoing isolated, length-modified and chemically-modified of crRNA and tracrRNA can be mixed in different combinations to form active crRNA:tracrRNA as the guide RNA for Cas9. For example, an isolated, length-modified tracrRNA can be combined with an isolated chemically-modified crRNA to form an active crRNA:tracrRNA as the guide RNA for Cas9. The examples provide illustrations of different combinations of isolated, length-modified and chemically-modified crRNA and tracrRNA resulting in active crRNA:tracrRNA as the guide RNA for Cas9.

The extent to which one needs particular chemically-modified nucleotides included in the isolated, length-modified and chemically-modified crRNA depends upon the application for which the resultant active crRNA:tracrRNA serves as the guide RNA for Cas9. In certain biochemical assays of the CRISPR-Cas endonuclease system, particularly where nucleases can be minimized or absent, one may not need extensively chemically-modified crRNA to effect robust activity of the resultant guide RNA for Cas9 of the CRISPR-Cas endonuclease system. This is attributed to the fact that chemically-modified nucleotides that confer resistance to nucleases are not necessary when nucleases are minimal or absent. Conversely in certain biochemical assays of the CRISPR-Cas endonuclease system, particularly use in certain cell lines having nuclease rich environments, one may need to chemically modify crRNA to effect robust activity of the resultant guide RNA for Cas9 of the CRISPR-Cas endonuclease system. In certain biological (in vivo) contexts, wherein a mixture including crRNA and tracrRNA is delivered to cells inside carrier vehicles, such as liposome nanoparticles, the isolated length-modified and chemically-modified crRNA and tracrRNA may require less extensive chemically-modified nucleotides than mixtures of crRNA and tracrRNA delivered directly into the blood stream or injected into organ systems as isolated, “naked,” RNA mixtures. The extent of chemical modification present in chemically-modified crRNA and tracrRNA can dictate the half-life of the relevant RNA molecules in vivo (that is, in the relevant biological context, such as, for example, in the blood stream or inside cells). Accordingly, the modification profile of chemically-modified crRNA and tracrRNA can be used to fine tune the biochemical and biological activity of the resultant crRNA:tracrRNA duplexes as a guide RNA for Cas9 in the CRISPR-Cas endonuclease system.

Although the prior art focuses on the structure of Cas9 as it interacts with a sgRNA, the disclosed design patterns described herein also contemplates the aforementioned crRNA:tracrRNA dual RNA systems. A single strand guide RNA offers several benefits, such as simplicity of a therapeutic design. However, standard solid phase phosphoramidite RNA synthesis shows diminishing yields for oligonucleotides as length increases and this problem becomes more apparent as length exceeds 60-70 bases. This precludes robust, cost-effective synthesis of some tracrRNAs as well as the chimeric sgRNA, especially at larger scales needed for some commercial or therapeutic applications. For this reason, the invention contemplates embodiments of not only sgRNA, but also alternate dual crRNA:tracrRNA as the guide RNA for Cas9. However, an isolated guide RNA having robust activity when combined with Cas9 in the CRISPR-Cas endonuclease system can be engineered by linkage or synthesis of appropriate crRNA and tracrRNA as an artificial, unimolecular sgRNA based upon the isolated, length-modified and chemically-modified forms of crRNA and tracrRNA provided herein. Long single guides of this type may be obtained by direct synthesis or by post-synthetic chemical conjugation of shorter strands.

The design of length-modified and chemically-modified crRNA compositions addresses the potential synthetic issues associated with crRNA oligonucleotides that are >40 nucleotides in length or with sgRNA oligonucleotides that are >80 nucleotides in length. The coupling efficiency of 2′-OMe-modified RNA monomers (effectively containing a protecting group on the 2′-OH) is greater than RNA monomer coupling. Incorporating 2′-OMe modified RNAs provides some advantages. First, it allows for longer oligonucleotides to be synthesized as either full 2′-OMe or RNA/2′-OMe mixed oligonucleotides. Secondly, the methods and compositions of the invention lead to synthesis and transfection of crRNA:tracrRNA that can evade detection by the immune system. It is well known that exogenous, unmodified RNAs trigger an innate immune response in mammalian cells as well as whole animals. Using 2′OMe-modified and/or LNA modified oligonucleotides can confer RNA stability to nucleases (a third advantage) as well as reduce cell death and toxicity associated with immunogenic triggers. These advantages are not unique to 2′-OMe modification or LNA modification, per se, as the other disclosed modified nucleotides having different chemical moieties (for example, 2′F, other 2′O-alkyls, and other bicyclic nucleotides) can offer similar benefits and advantages in terms of conferring resistance to nucleases.

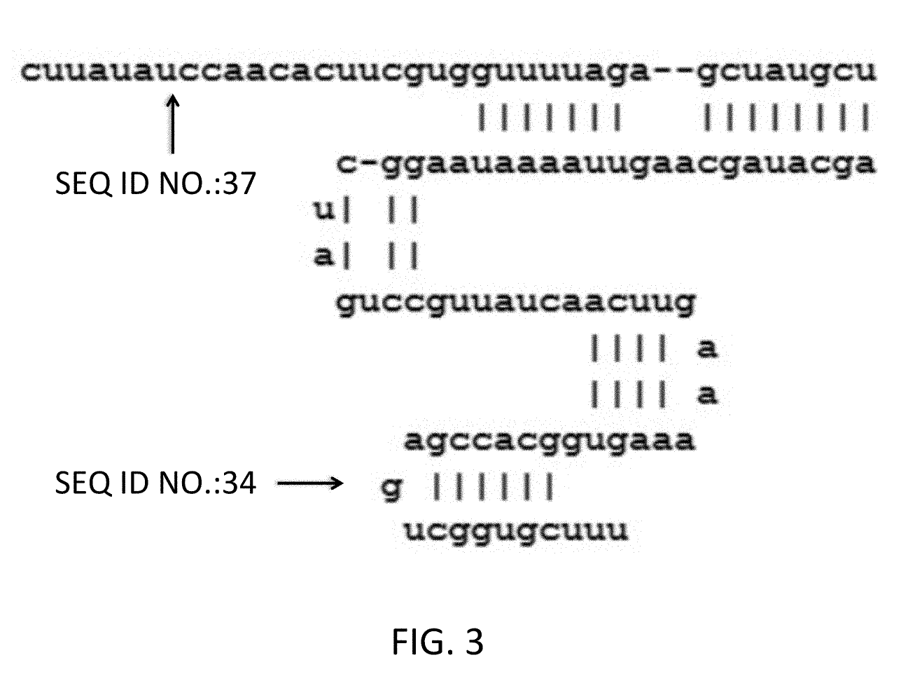

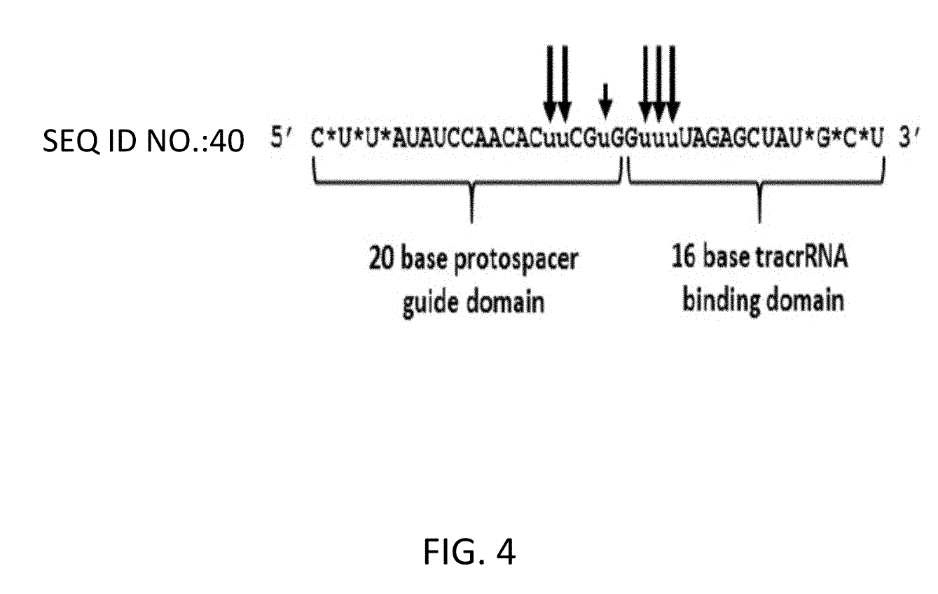

In another embodiment, an isolated crRNA of formula (I) is designed with modifications that are empirically determined. As depicted in FIG. 3, the 12 nucleotides at the 3′-end of the Z domain (the tracrRNA-binding domain) and the 10-12 nucleotides at the 5′-end of the X domain (within the protospacer domain) represent universal nucleotides amenable to substitution with chemically-modified nucleotides, wherein the resultant RNAs retain robust activity in the CRISPR-Cas endonuclease system. Yet other nucleotides within the 5′-end of the Z domain (the tracrRNA-binding domain) are intolerant to substitution with chemically-modified nucleotides (FIG. 4). Yet the ability of other sites within an isolated crRNA of formula (I) to accept chemically-modified nucleotides and retain activity in the CRISPR-Cas endonuclease system is largely determined empirically. The tracrRNA binding domain (Z domain) of the crRNA is constant (i.e., sequence does not change as target site varies), so the modifications patterns described herein are universal to all crRNAs regardless of target site and can be broadly applied. The protospacer (X domain) of the crRNA varies with target, and the tolerance of some of the base positions within this domain to chemical modification vary with sequence context and, if maximal chemical modification of a site is desired, may benefit from empiric optimization. However, some of the residues within the target-specific protospacer (X) domain can be modified without consideration to sequence context. The 10-12 residues at the 5′-end of this domain can be substituted with 2′-modified residues with the expectation that full activity of the modified crRNA will be maintained.

The applications of Cas9-based tools are many and varied. They include, but are not limited to: plant gene editing, yeast gene editing, rapid generation of knockout/knockin animal lines, generating an animal model of disease state, correcting a disease state, inserting a reporter gene, and whole genome functional screening.

The utility of the present invention is further expanded by including mutant versions of Cas enzymes, such as a D10A and H840a double mutant of Cas9 as a fusion protein with transcriptional activators (CRISPRa) and repressors (CRISPRi) (see Xu, T. et al., Appl Environ Microbiol, 2014. 80(5): p. 1544-52). The Cas9-sgRNA complex also can be used to target single-stranded mRNA as well (see 0′ Connell, M. R. et al., Nature, 516:263, 2014). In the same way as targeting dsDNA, crRNA:tracrRNA can be used with a PAMmer DNA oligonucleotide to direct Cas9 cleavage to the target mRNA or use it in the mRNA capture assay described by O'Connell.

By utilizing an approach to deliver synthetic RNA oligonucleotides for CRISPR/Cas9 applications, it is possible to 1) use mass spectroscopy to confirm discrete RNA sequences, 2) selectively insert 2′-OMe modified RNAs in well-tolerated locations to confer stability and avoid immunogenicity yet retain functional efficacy, 3) selectively insert LNA modified nucleotides in well tolerated locations to confer stability and avoid immunogenicity yet retain functional efficacy, 4) specifically control the amount of RNA that is introduced into cells for a controlled transient effect, and 5) eliminate concern over introducing dsDNA that would be endogenously transcribed to RNA but could also become substrate in either homology-directed repair pathway or in non-homologous end joining resulting in an integration event. These integration events can lead to long term undesired expression of crRNA or tracrRNA elements. Further, integration can disrupt other genes in a random and unpredictable fashion, changing the genetic material of the cell in undesired and potentially deleterious ways. The present invention is therefore desirable as a means to introduce transient expression of elements of the CRISPR pathway in cells in a way which is transient and leaves no lasting evidence or change in the genome outside of whatever alteration is intended as directed by the crRNA guide.

CRISPR-Cas Endonuclease Systems

A competent CRISPR-Cas endonuclease system includes a ribonucleoprotein (RNP) complex formed with isolated Cas9 protein and isolated guide RNA selected from one of a dual crRNA:tracrRNA combination and a chimeric sgRNA. In some embodiments, isolated length-modified and/or chemically-modified forms of crRNA and tracrRNA are combined with purified Cas9 protein or Cas9 mRNA.

Applications

In a first aspect, an isolated crRNA comprising a length-modified and chemically modified form of formula (I) is provided:

5′-X—Z-3′ (I).

X is a target-specific protospacer domain and Z is a tracrRNA-binding domain. The tracrRNA binding domain further comprises at least one chemically modified nucleotide. The isolated crRNA is active in a Clustered Regularly Interspaced Short Palindromic Repeats (CRISPR)/CRISPR-associated protein endonuclease system. In a first respect, the protospacer domain consists of 17, 18, 19, or 20 nucleotides. In a second respect, the at least one chemically modified nucleotide is at or near the 3′ end. In one embodiment, the at least one chemically modified nucleotide consists of, 2-O-Methyl modifications, phosphorothioate internucleotide linkages, locked nucleic acids, or a combination. In another embodiment, the tracrRNA-binding domain is selected from the group consisting of SEQ ID No. 41, SEQ ID No. 42, SEQ ID No. 43 and SEQ ID No. 44.

In a second aspect, a method of performing gene editing is provided. The method includes a step of contacting a candidate editing target site locus with an active CRISPR/Cas endonuclease system having a suitable crRNA. The crRNA has a tracrRNA binding domain. The tracrRNA binding domain further comprises at least one chemically modified nucleotide. In a first respect, the tracrRNA binding domain is selected form the group consisting of SEQ ID No. 41, SEQ ID No. 42, SEQ ID No. 43 and SEQ ID No. 44.

In a third aspect, a method of performing gene editing is provided. The method includes the step of contacting a candidate editing target site locus in bacteria with an active CRISPR/Cas endonuclease system having a suitable crRNA. The crRNA has a tracrRNA binding domain. The tracrRNA binding domain further comprises at least one chemically modified nucleotide. In a first respect, the tracrRNA binding domain is selected form the group consisting of SEQ ID No. 46.

### EXAMPLES

### Example 1

This examples illustrates functioning of chemically modified and truncated crRNAs to direct genome editing in mammalian cells.

The crRNA and tracrRNA oligonucleotides were synthesized having various chemical modifications relative to the truncated sequences as indicated (Table 1).

TABLE 1
crRNA and tracrRNA pairs for use in in vivo biochemical studies of cleavage
of the HPRT1 target DNA by Cas9 endonuclease.

| cr/tracr | | | |
|---|---|---|---|
| RNA | SEQ ID | crRNA Sequence | |
| Pair | No. | tracrRNA Sequence | Length |
| unmod/ | 1 | cuuauauccaacacuucgugguuuuagagcuaugcu | 36 |
| unmod | 34 | agcauagcaaguuaaaauaaggcuaguccguuaucaacuugaaaaaguggcaccgagu | 67 |
| | | cggugcuuu | |
| Alt- | 2 | C3-cuuauauccaacacuucgugguuuuagagcuaugcu-C3 | 36 |
| R/mod | 33 | a*g*cauagcaaguuaaaauaaggcuaguccguuaucaacuugaaaaaguggcaccga | 67 |
| | | gucggugcu*u*u | |
| LNA | 3 | c*u*uauauccaacacuucgugguuuuagagcuau+g*+c*u | 36 |
| mod1/ | 33 | a*g*cauagcaaguuaaaauaaggcuaguccguuaucaacuugaaaaaguggcaccga | 67 |
| mod | | gucggugcu*u*u | |
| heavy | 4 | c*u*u*auauccaacacuucgugguuuuagagcuau*g*c*u | 36 |
| mod/ | 33 | a*g*cauagcaaguuaaaauaaggcuaguccguuaucaacuugaaaaaguggcaccga | 67 |
| mod | | gucggugcu*u*u | |

Oligonucleotide sequences are shown 5′-3′. Lowercase = RNA, Underline = 2′-O-methyl RNA, * = phosphorothioate, +a, +c, +t, +g = LNA; C3 = C3 spacer (propanediol modifier). Lengths of RNA oligonucleotides are indicated (bases).

The crRNA contained a 20 base protospacer guide sequence complementary to a site in the human HPRT1 gene adjacent to a suitable “NGG” PAM site. The crRNA and tracrRNA pairs were tested for the ability to direct cleavage of the target sequence in HEK293 cells.

The crRNA and tracrRNA were annealed in Duplex Buffer at a 1:1 molar ratio. The duplexed crRNA:tracrRNA were incubated with Alt-R® wild type (WT) Cas9 protein (Integrated DNA Technologies) for 10 minutes at room temperature at a 1.2:1 molar ratio to form the ribonucleoprotein complex (RNP). RNP complexes were delivered into HEK293 cells via Amaxa Nucleofection (Lonza; program: DS-150, buffer=SF) at 4 μM, 2 μM, 0.5 μM, or 0.25 μM (protein concentration given, gRNA concentration is 1.2×) in the presence of 4 μM Alt-R Electroporation Enhancer (Integrated DNA Technologies) (4 μM for all doses). Genomic DNA was isolated after 48 hours using QuickExtract (Epicentre) and HPRT region of interest amplified with KAPA HiFi Polymerase (Kapa Biosystems). Heteroduplexes were formed by heating amplicons to 95° C. and slowly cooling to room temperature. Heteroduplexes were digested with 2 units of T7EI (IDT Alt-R Genome Editing Kit) at 37° C. for 60 min. Digested samples were analyzed for total editing by visualization on the Fragment Analyzer (Advanced Analytical).

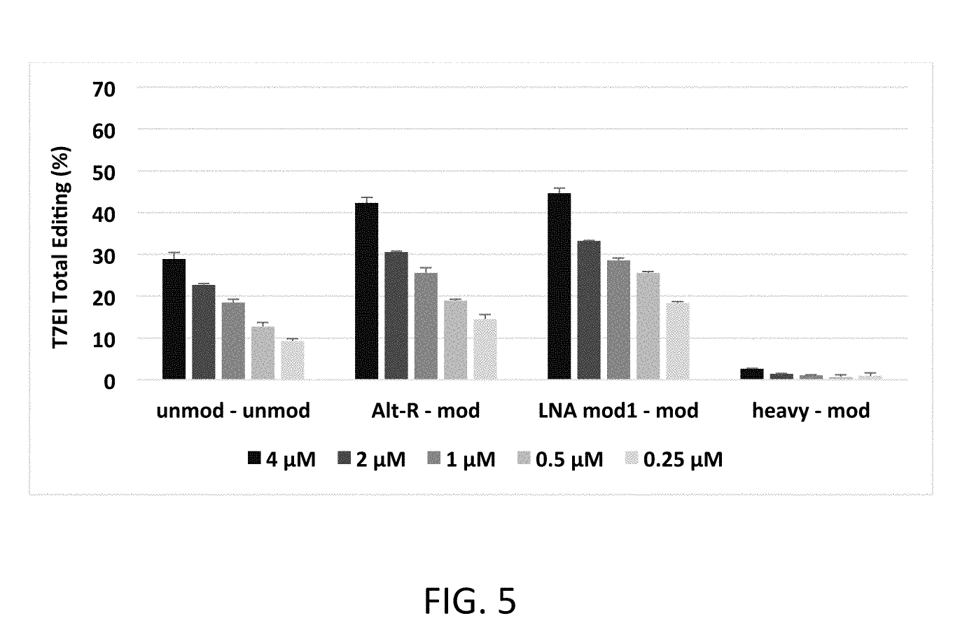

Native wild-type (WT) crRNAs have a 19-20 base protospacer domain (guide, which binds to a target nucleic acid) at the 5′-end and a 22 base domain at the 3′-end that binds to the tracrRNA. Thus WT crRNAs are 41-42 bases long. The WT tracrRNA is 89 bases long. It was observed that unmodified truncated versions of the crRNA and tracrRNA are also effective (unmod/unmod crRNA/tracrRNA pair). A 36 base crRNA consisting of a 20 base protospacer and a 16 base tracrRNA binding domain (SEQ ID NO. 1) complexed with a 67 base tracrRNA (SEQ ID No. 34) supported cleavage of the target sequence. See FIG. 5. These findings are significant as it permits use of shorter RNA components to direct Cas9 target recognition and cleavage. Shorter RNA oligonucleotides are less expensive and less difficult for chemical synthesis, requiring less purification and giving higher yields than longer RNA oligonucleotides.

Some of the elements of the truncated crRNA and truncated tracrRNA were further chemically modified. The Alt-R/mod cr/tracrRNA pair demonstrate the usage of C3 spacers at the 5′ and 3′ end of the crRNA (SEQ ID No. 2) as well as modifications of the tracrRNA comprising 2′-O-methyl RNA and phosphorothioate linkages (SEQ ID No. 33). As shown in FIG. 5 (Alt-R-mod) these chemical modifications support increased cleavage of the target region.

Additional modifications to the truncated crRNA and truncated tracrRNA can direct cleavage. The LNA mod1 crRNA/modified tracrRNA pair demonstrate the usage of LNA modified nucleotides. In addition to 2′-O-methyl RNA and phosphorothioate linkages, LNA modified nucleotides were incorporated into the crRNA (SEQ ID No. 3). As shown in FIG. 5 LNA modified crRNA are capable of directing increased genome editing at lower doses.

This example demonstrates that for the purposes of gene editing in mammalian cells truncated versions of crRNA and tracrRNA are tolerated and the total genome editing can be improved with additional chemical modifications to the RNA sequence. Furthermore, this example demonstrates that the inclusion of LNA nucleotides in the crRNA does not negatively impact the function of the crRNA/tracrRNA complex to direct genome editing.

In summary, this example demonstrates that the inclusion of LNA modified nucleotides in the crRNA (SEQ ID No. 3) retains high functional activity. Use of LNA modified nucleotides in crRNA can further increase the on-target activity especially when delivered at lower doses.

### Example 2

Example 1 demonstrates that LNA containing crRNA can show higher functional activity in mammalian gene editing. The present example shows further optimization of the placement of LNA modified nucleotides in the crRNA.

A series of crRNA and tracrRNA oligonucleotides were synthesized having varied LNA nucleotide placements (Table 2).

TABLE 2
crRNA and tracrRNA pairs for use in in vivo biochemical studies of cleavage
of the HPRT1 target DNA by Cas9 endonuclease.

| cr/tracr | | | |
|---|---|---|---|
| RNA | SEQ ID | crRNA Sequence | |
| Pair | No. | tracrRNA Sequence | Length |
| Alt- | 2 | C3-cuuauauccaacacuucgugguuuuagagcuaugcu-C3 | 36 |
| R/mod | 33 | a*g*cauagcaaguuaaaauaaggcuaguccguuaucaacuugaaaaaguggcaccga | 67 |
| | | gucggugcu*u*u | |
| LNA | 3 | c*u*uauauccaacacuucgugguuuuagagcuau+g*+c*u | 36 |
| mod1/ | 33 | a*g*cauagcaaguuaaaauaaggcuaguccguuaucaacuugaaaaaguggcaccga | 67 |
| mod | | gucggugcu*u*u | |
| LNA | 5 | c*u*uauauccaacacuucgugguuuuagagcua+t+g*+c*u | 36 |
| mod2/ | 33 | a*g*cauagcaaguuaaaauaaggcuaguccguuaucaacuugaaaaaguggcaccga | 67 |
| mod | | gucggugcu*u*u | |
| LNA | 6 | c*u*uauauccaacacuucgugguuuuaga+g+cuau+g*+c*u | 36 |
| mod3/ | 33 | a*g*cauagcaaguuaaaauaaggcuaguccguuaucaacuugaaaaaguggcaccga | 67 |
| mod | | gucggugcu*u*u | |
| LNA | 7 | c*u*uauauccaacacuucgugguuuuaga+g+cuau+g*+c*u | 36 |
| mod4/ | 33 | a*g*cauagcaaguuaaaauaaggcuaguccguuaucaacuugaaaaaguggcaccga | |
| mod | | gucggugcu*u*u | 67 |

Oligonucleotide sequences are shown 5′-3′. Lowercase = RNA, Underline = 2′-O-methyl RNA, * = phosphorothioate, +a, +c, +t, +g = LNA; C3 = C3 spacer (propanediol modifier). Lengths of RNA′ oligonucleotides are indicated (bases).′

The crRNA contained a 20 base protospacer guide sequence complementary to a site in the human HPRT1 gene adjacent to a suitable “NGG” PAM site. The crRNA and tracrRNA pairs were tested for the ability to direct cleavage of the target sequence in HEK293 cells.

The crRNA and tracrRNA were annealed in Duplex Buffer at a 1:1 molar ratio. The duplexed crRNA:tracrRNA were incubated with Alt-R WT Cas9 protein (Integrated DNA Technologies) for 10 minutes at room temperature at a 1.2:1 molar ratio to form the ribonucleoprotein complex (RNP). RNP complexes were delivered into HEK293 cells via Amaxa Nucleofection (Lonza; program: DS-150, buffer=SF) at 4 μM, 2 μM, or 0.5 (protein concentration given, gRNA concentration is 1.2×) in the presence of 4 μM Alt-R Electroporation Enhancer (Integrated DNA Technologies) (4 μM for all doses). Genomic DNA was isolated after 48 hours using QuickExtract (Epicentre) and HPRT region of interest amplified with KAPA HiFi Polymerase (Kapa Biosystems). Heteroduplexes were formed by heating amplicons to 95° C. and slowly cooling to room temperature. Heteroduplexes were digested with 2 units of T7EI (IDT Alt-R Genome Editing Kit) at 37° C. for 60 min. Digested samples were analyzed for total editing by visualization on the Fragment Analyzer (Advanced Analytical).

Some kind of chemical modification is usually necessary for synthetic nucleic acids to function well in an intracellular environment due to the presence of exonucleases and endonucleases that degrade unmodified oligonucleotides. A wide range of modifications have been described that confer nuclease resistance to oligonucleotides. The precise combination and order of modifications employed that works well for a given application can vary with sequence context and the nature of the protein interactions required for biological function. Extensive prior work has been done relating to chemical modification of antisense oligonucleotides (which interact with RNase H1) and siRNAs (which interact with DICER, AGO2, and other proteins). It is expected that chemical modification will improve function of the CRISPR crRNA:tracrRNA complex. However, it is not possible to predict what modifications and/or pattern of modifications will be compatible for association of the RNAs with Cas9 in a functional way. The present invention defines chemical modification patterns for the crRNA that retain high levels of function to direct Cas9 mediated gene editing in mammalian cells. The survey in Example 2 was performed targeting a single site in the human HPRT1 gene. Note that modification patterns of the 20 base 5′-end protospacer guide domain of the crRNA that perform well may vary with sequence context.

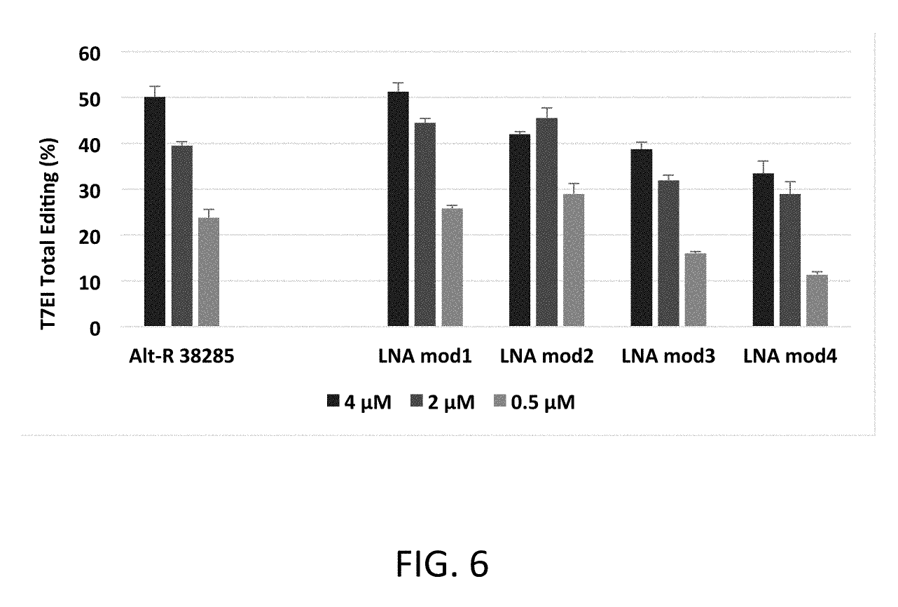

In general, modification of the crRNA had a small impact on gene editing efficiency when the RNAs were transfected at high dose where the RNAs are present in excess. At lower doses, the modified reagents retained potency. FIG. 6 shows a plot of the functional gene editing observed using the T7E1 assay in HEK293 cells using chemically modified crRNA (SEQ ID Nos. 3, and 4-7) duplexed with tracrRNA (SEQ ID No. 33) tested at varied input concentrations.

All of the compounds studied directed CRISPR/Cas editing at the HPRT1 locus in HEK293 cells. Additionally, all compounds studied directed CRISPR/Cas editing at the HPRT1 locus with various concentrations of duplexed crRNA/tracrRNA. Efficiency of editing at a concentration of 4 μM varied from 33% to 51%. Efficiency of editing at a concentration of 2 μM varied from 28% to 44%. Efficiency of editing at a concentration of 0.5 μM varied from 11% to 25%. The most effective crRNA/tracrRNA combination was the combination of LNA mod 1 (SEQ ID No. 3) with modified tracrRNA (SEQ ID No. 33). A plot of editing efficiency for each LNA mod pattern is shown in FIG. 6 and Table 3 shows the editing efficiency for each crRNA tested.

TABLE 3
Editing efficiency percentage of the modified crRNA tested
at the corresponding concentration all paired
with modified tracrRNA (SEQ ID 34).

| | SEQ ID | Concentration | | | Std Dev | | |
|---|---|---|---|---|---|---|---|
| crRNA | No. | 4 μM | 2 μM | 0.5 μM | 4 μM | 2 μM | 0.5 μM |
| Alt-R 38285 | 2 | 50.18 | 39.51 | 23.81 | 2.23 | 0.88 | 1.82 |
| LNA mod1 | 3 | 51.27 | 44.46 | 25.81 | 1.96 | 0.94 | 0.66 |
| LNA mod2 | 5 | 42.02 | 45.57 | 28.97 | 0.58 | 2.16 | 2.26 |
| LNA mod3 | 6 | 38.72 | 31.90 | 15.98 | 1.52 | 1.19 | 0.47 |
| LNA mod4 | 7 | 33.46 | 28.98 | 11.37 | 2.72 | 2.62 | 0.66 |

The plot shown in FIG. 6 demonstrates that LNA modifications can be placed in varied positions across the crRNA and that editing efficiency is comparable to, if not better than, crRNA compositions containing only end-blocking modifications. Additionally, the plot shown in FIG. 6 demonstrates that LNA-modified crRNA can function to direct CRISPR/Cas editing at varied input concentrations and that even at reduced concentrations the modified crRNAs are effective to direct CRISPR/Cas editing.

### Example 3

The present example demonstrates that LNA modified nucleotides in crRNA are effective to direct CRISPR/Cas editing at various target genomic positions. Additionally, the present example demonstrates that a chemically modified 2 part crRNA/tracrRNA complex has similar editing efficiency to a chemically modified sgRNA.

A series of crRNAs (Table 4) targeting different genomic positions were made. The crRNAs were either C3 modified (SEQ ID Nos. 2, 10, 14, 18, 22, or 26), LNA mod1 pattern (SEQ ID Nos. 3, 11, 15, 19, 23, or 27), LNA mod2 pattern (SEQ ID Nos. 5, 12, 16, 20, 24, or 28), or a modified sgRNA (SEQ ID Nos. 9, 13, 17, 21, 25, or 29). All crRNAs were duplexed with modified tracrRNA (SEQ ID No. 33).

TABLE 4
crRNA and tracrRNA pairs for use in in vivo biochemical studies of cleavage of
different target DNA by Cas9 endonuclease.

| cr/tracr | | | | |
|---|---|---|---|---|
| RNA | SEQ ID | crRNA Sequence | | Target |
| Pair | No. | tracrRNA Sequence | Length | Site |
| min | 2 | C3-cuuauauccaacacuucgugguuuuagagcuaugcu-C3 | 36 | HPRT |
| mod/ | 33 | a*g*cauagcaaguuaaaauaaggcuaguccguuaucaacuugaaaaaguggc | 67 | 38285 |
| mod | | accgagucggugcu*u*u | | |
| LNA | 3 | c*u*uauauccaacacuucgugguuuuagagcuau+g*+c*u | 36 | HPRT |
| mod1/ | 33 | a*g*cauagcaaguuaaaauaaggcuaguccguuaucaacuugaaaaaguggc | 67 | 38285 |
| mod | | accgagucggugcu*u*u | | |
| LNA | 5 | c*u*uauauccaacacuucgugguuuuagagcua+t+g*+c*u | 36 | HPRT |
| mod2/ | 33 | a*g*cauagcaaguuaaaauaaggcuaguccguuaucaacuugaaaaaguggc | 67 | 38285 |
| mod | | accgagucggugcu*u*u | | |
| sgRNA | 9 | c*u*u*auauccaacacuucgugguuuuagagcuagaaauagcaaguuaaaa | 100 | HPRT |
| | | uaaggcuaguccguuaucaacuugaaaaaguggcaccgagucggugcu*u*u* | | 38285 |
| | | u | | |
| min | 10 | C3-gaggcuauucugcccauuugguuuuagagcuaugcu-C3 | 36 | Myc 459 |
| mod/ | 33 | a*g*cauagcaaguuaaaauaaggcuaguccguuaucaacuugaaaaaguggc | 67 | |
| mod | | accgagucggugcu*u*u | | |
| LNA | 11 | g*a*ggcuauucugcccauuugguuuuagagcuau+g*+c*u | 36 | Myc 459 |
| mod1/ | 33 | a*g*cauagcaaguuaaaauaaggcuaguccguuaucaacuugaaaaaguggc | 67 | |
| mod | | accgagucggugcu*u*u | | |
| LNA | 12 | g*a*ggcuauucugcccauuugguuuuagagcua+t+g*+c*u | 36 | Myc 459 |
| mod2/ | 33 | a*g*cauagcaaguuaaaauaaggcuaguccguuaucaacuugaaaaaguggc | 67 | |
| mod | | accgagucggugcu*u*u | | |
| sgRNA | 13 | g*a*ggcuauucugcccauuugguuuuagagcuagaaauagcaaguuaaaau | 100 | Myc 459 |
| | | aaggcuaguccguuaucaacuugaaaaaguggcaccgagucggugcu*u*u*u | | |
| Hamp | 14 | C3-uggcacugagcucccagaucguuuuagagcuaugcu-C3 | 36 | Hamp |
| 253 min | 33 | a*g*cauagcaaguuaaaauaaggcuaguccguuaucaacuugaaaaaguggc | 67 | 253 |
| mod/ | | accgagucggugcu*u*u | | |
| mod | | | | |
| LNA | 15 | u*g*gcacugagcucccagaucguuuuagagcuau+g*+c*u | 36 | Hamp |
| mod1/ | 33 | a*g*cauagcaaguuaaaauaaggcuaguccguuaucaacuugaaaaaguggc | 67 | 253 |
| mod | | accgagucggugcu*u*u | | |
| LNA | 16 | u*g*gcacugagcucccagaucguuuuagagcua+t+g*+c*u | 36 | Hamp |
| mod2/ | 33 | a*g*cauagcaaguuaaaauaaggcuaguccguuaucaacuugaaaaaguggc | 67 | 253 |
| mod | | accgagucggugcu*u*u | | |
| sgRNA | 17 | u*g*gcacugagcucccagaucguuuuagagcuagaaauagcaaguuaaaaua | 100 | Hamp |
| | | aggcuaguccguuaucaacuugaaaaaguggcaccgagucggugcu*u*u*u | | 253 |
| min | 18 | C3-aggacaaguucucugaguucguuuuagagcuaugcu-C3 | 36 | Apoc3 |
| mod/ | 33 | a*g*cauagcaaguuaaaauaaggcuaguccguuaucaacuugaaaaaguggc | 67 | 2929 |
| mod | | accgagucggugcu*u*u | | |
| LNA | 19 | a*g*gacaaguucucugaguucguuuuagagcuau+g*+c*u | 36 | Apoc3 |
| mod1/ | 33 | a*g*cauagcaaguuaaaauaaggcuaguccguuaucaacuugaaaaaguggc | 67 | 2929 |
| mod | | accgagucggugcu*u*u | | |
| LNA | 20 | a*g*gacaaguucucugaguucguuuuagagca+t+g*+c*u | 36 | Apoc3 |
| mod2/ | 33 | a*g*cauagcaaguuaaaauaaggcuaguccguuaucaacuugaaaaaguggc | 67 | 2929 |
| mod | | accgagucggugcu*u*u | | |
| sgRNA | 21 | a*g*gacaaguucucugaguucguuuuagagcuagaaauagcaaguuaaaau | 100 | Apoc3 |
| | | aaggcuaguccguuaucaacuugaaaaaguggcaccgagucggugcu*u*u*u | | 2929 |
| min | 22 | C3-ccccuccaaccuggaauuccguuuuagagcuaugcu-C3 | 36 | Serpina |
| mod/ | 33 | a*g*cauagcaaguuaaaauaaggcuaguccguuaucaacuugaaaaaguggc | 67 | 1130 |
| mod | | accgagucggugcu*u*u | | |
| LNA | 23 | c*c*ccuccaaccuggaauuccguuuuagagcuau+g*+c*u | 36 | Serpina |
| mod1/ | 33 | a*g*cauagcaaguuaaaauaaggcuaguccguuaucaacuugaaaaaguggc | 67 | |
| mod | | accgagucggugcu*u*u | | |
| LNA | 24 | c*c*ccuccaaccuggaauuccguuuuagagcua+t+g*+c*u | 36 | Serpina |
| mod2/ | 33 | a*g*cauagcaaguuaaaauaaggcuaguccguuaucaacuugaaaaaguggc | 67 | 1130 |
| mod | | accgagucggugcu*u*u | | |
| sgRNA | 25 | c*c*ccuccaaccuggaauuccguuuuagagcuagaaauagcaaguuaaaaua | 100 | Serpina |
| | | aggcuaguccguuaucaacuugaaaaaguggcaccgagucggugcu*u*u*u | | 1130 |
| min | 26 | C3-gcugcuguagcugauuccauguuuuagagcuaugcu-C3 | 36 | Stat3 |
| mod/ | 33 | a*g*cauagcaaguuaaaauaaggcuaguccguuaucaacuugaaaaaguggc | 67 | 39988 |
| mod | | accgagucggugcu*u*u | | |
| LNA | 27 | g*c*ugcuguagcugauuccauguuuuagagcuau+g*+c*u | 36 | Stat3 |
| mod1/ | 33 | a*g*cauagcaaguuaaaauaaggcuaguccguuaucaacuugaaaaaguggc | 67 | 39988 |
| mod | | accgagucggugcu*u*u | | |
| LNA | 28 | g*c*ugcuguagcugauuccauguuuuagagcua+t+g*+c*u | 36 | Stat3 |
| mod2/ | 33 | a*g*cauagcaaguuaaaauaaggcuaguccguuaucaacuugaaaaaguggc | 67 | 39988 |
| mod | | accgagucggugcu*u*u | | |
| sgRNA | 29 | g*c*ugcuguagcugauuccauguuuuagagcuagaaauagcaaguuaaaau | 100 | Stat3 |
| | | aaggcuaguccguuaucaacuugaaaaaguggcaccgagucggugcu*u*u*u | | 39988 |

Oligonucleotide sequences are shown 5′-3′. Lowercase = RNA, Underline = 2′-O-methyl RNA, * = phosphorothioate, +a, +c, +t, +g = LNA; C3 = C3 spacer (propanediol modifier). Lengths of RNA oligonucleotides are indicated (bases).

The crRNA contained a 20 base protospacer guide sequence complementary to different target sites. The genomic loci tested were in the human genes HPRT1, MYC, HAMP, APOC3, SERPINA1, or STAT3. All target regions of the genes were adjacent to a suitable “NGG” PAM site. The crRNA and tracrRNA pairs were tested for the ability to direct cleavage of the target sequence in HEK293 cells.

The crRNA and tracrRNA were annealed in Duplex Buffer at a 1:1 molar ratio. The duplexed crRNA:tracrRNA or sgRNA were incubated with Alt-R® wild type Cas9 protein (Integrated DNA Technologies) for 10 minutes at room temperature at a 1.2:1 molar ratio to form the ribonucleoprotein complex (RNP). RNP complexes were delivered into HEK293 cells via Amaxa Nucleofection (Lonza; program: DS-150, buffer=SF) at 4 or 0.5 (protein concentration given, gRNA concentration is 1.2×) in the presence of 4 μM Alt-R Electroporation Enhancer (Integrated DNA Technologies) (4 μM for all doses). Genomic DNA was isolated after 48 hours using QuickExtract (Epicentre) and HPRT region of interest amplified with KAPA HiFi Polymerase (Kapa Biosystems). Heteroduplexes were formed by heating amplicons to 95° C. and slowly cooling to room temperature. Heteroduplexes were digested with 2 units of T7EI (IDT Alt-R Genome Editing Kit) at 37° C. for 60 min. Digested samples were analyzed for total editing by visualization on the Fragment Analyzer (Advanced Analytical).

TABLE 5
Editing efficiency percentage of the modified crRNA or
sgRNA tested at the corresponding concentration.

| | | | Concentration | | Std Dev | |
|---|---|---|---|---|---|---|
| Target | crRNA | SEQ ID No | 4 μM | 0.5 μm | 4 μM | 0.5 μM |
| HPRT | Min Mod | 2 | 44.70 | 35.16 | 0.43 | 3.07 |
| 38285 | | | | | | |
| HPRT | LNA | 3 | 46.91 | 32.83 | 0.61 | 3.32 |
| 38285 | Mod1 | | | | | |
| HPRT | LNA | 5 | 31.82 | 22.77 | 0.70 | 2.78 |
| 38285 | Mod2 | | | | | |
| HPRT | sgRNA | 9 | 48.17 | 43.08 | 1.14 | 5.30 |
| 38285 | | | | | | |
| Myc459 | Min Mod | 10 | 62.07 | 47.87 | 1.42 | 0.95 |
| Myc459 | LNA | 11 | 78.71 | 73.73 | 0.92 | 1.34 |
| | Mod1 | | | | | |
| Myc459 | LNA | 12 | 79.46 | 76.41 | 1.45 | 1.97 |
| | Mod2 | | | | | |
| Myc459 | sgRNA | 13 | 81.74 | 81.08 | 1.14 | 2.05 |
| Hamp 253 | Min Mod | 14 | 22.77 | 8.42 | 1.87 | 5.96 |
| Hamp 253 | LNA | 15 | 49.55 | 26.99 | 0.90 | 0.81 |
| | Mod1 | | | | | |
| Hamp 253 | LNA | 16 | 36.43 | 21.19 | 0.68 | 0.76 |
| | Mod2 | | | | | |
| Hamp 253 | sgRNA | 17 | 67.66 | 44.17 | 1.02 | 0.17 |
| Apoc3 | Min Mod | 18 | 21.87 | 9.60 | 0.76 | 1.03 |
| Apoc3 | LNA | 19 | 50.68 | 21.03 | 1.97 | 1.55 |
| | Mod1 | | | | | |
| Apoc3 | LNA | 20 | 45.60 | 19.00 | 2.87 | 1.74 |
| | Mod2 | | | | | |
| Apoc3 | sgRNA | 21 | 39.09 | 13.96 | 2.22 | 0.94 |
| Serpina1 | Min Mod | 22 | 63.58 | 45.32 | 3.17 | 3.40 |
| 130 | | | | | | |
| Serpina1 | LNA | 23 | 67.16 | 57.33 | 2.51 | 3.78 |
| 130 | Mod1 | | | | | |
| Serpina1 | LNA | 24 | 67.37 | 53.46 | 4.38 | 1.65 |
| 130 | Mod2 | | | | | |
| Serpina1 | sgRNA | 25 | 64.42 | 62.17 | 3.72 | 1.41 |
| 130 | | | | | | |
| Stat3 39988 | Min Mod | 26 | 56.72 | 27.03 | 3.04 | 2.97 |
| Stat3 39988 | LNA | 27 | 63.15 | 39.13 | 1.95 | 4.75 |
| | Mod1 | | | | | |
| Stat3 39988 | LNA | 28 | 38.29 | 18.08 | 1.73 | 1.52 |
| | Mod2 | | | | | |
| Stat3 39988 | sgRNA | 29 | 66.81 | 51.72 | 0.76 | 5.31 |

The survey in Example 3 was performed targeting different sites in the human genome. The targeted sites included HPRT1, MYC, HAMP, APOC3, SERPINA1, and STAT3. Note that modification patterns of the 20 base 5′-end protospacer guide domain of the crRNA that perform well may vary with sequence context.

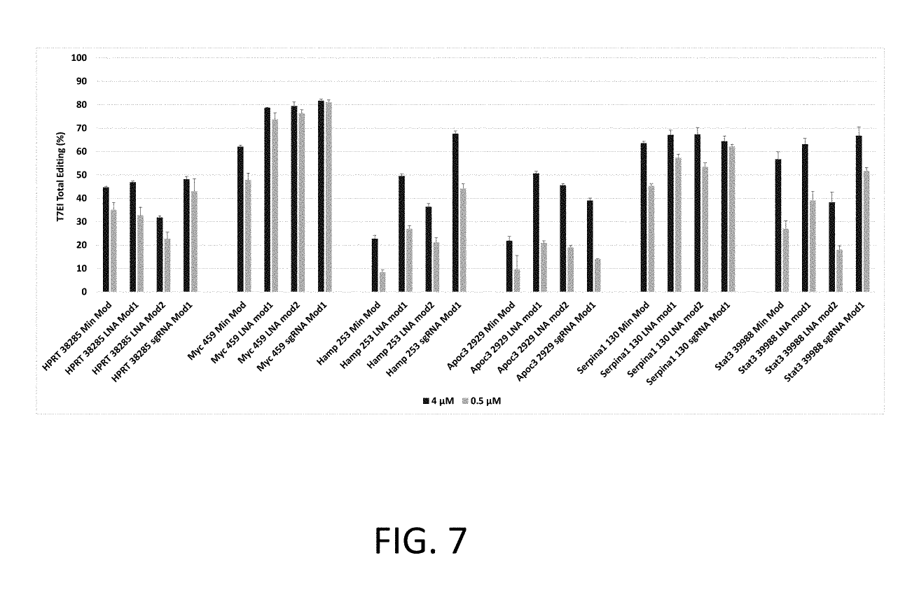

In general, modification of the crRNA had a small impact on gene editing efficiency when the RNAs were transfected at high dose where the RNAs are present in excess. At lower doses, the modified reagents retained potency. The degree of improvement varied with site. FIG. 7 shows a plot of the functional gene editing observed using the T7E1 assay in HEK293 cells using LNA containing crRNAs (SEQ ID Nos. 3, 5, 11-12, 15-16, 19-20, 23-24, and 27-28) duplexed with tracrRNA (SEQ ID No. 33) tested at varied input concentrations compared to sgRNA (SEQ ID Nos. 9, 13, 17, 21, 25 and 29) and compared to min-mod crRNA patterns (SEQ ID Nos. 2, 10, 14, 18, 22, and 26) duplexed with tracrRNA (SEQ ID No. 33).

The LNA modified crRNAs were capable of directing cleavage at the desired genomic region in all 6 sites studied. Additionally, the LNA mod1 pattern had increased editing efficiency over the respective minimally modified crRNA. Additionally, the LNA mod1 crRNA pattern had similar editing efficiency as the respective sgRNA. The data also show that the LNA modified crRNA were capable of directing cleavage at reduced concentrations at levels similar to or better than min-mod crRNAs.

### Example 4

The present example demonstrates the use of chemically modified and truncated crRNA/tracrRNA complexes transfected with Cas9 mRNA. Furthermore, this example demonstrates the need for more highly modified crRNA/tracrRNA complexes when the Cas9 protein is delivered as mRNA which is subsequently expressed in the cell.

A series of crRNAs (Table 6) targeting the HPRT1 gene were made. The crRNAs were either unmodified (SEQ ID No.1), C3 modified (SEQ ID No. 2), med-mod (SEQ ID No. 31), or heavy mod (SEQ ID Nos. 32). The unmodified crRNA was duplexed with an unmodified tracrRNA (SEQ ID No 34). All modified crRNAs were duplexed with modified tracrRNA (SEQ ID No. 33).

TABLE 6
crRNA and tracrRNA pairs for use in in vivo biochemical studies of cleavage of
the HPRT1 target DNA by transfected Cas9 mRNA.

| cr/tracr | | | | |
|---|---|---|---|---|
| RNA | SEQ ID | crRNA Sequence | | Target |
| Pair | No. | tracrRNA Sequence | Length | Site |
| Unmod/ | 1 | cuuauauccaacacuucgugguuuuagagcuaugcu | 36 | HPRT |
| unmod | 34 | agcauagcaaguuaaaauaaggcuaguccguuaucaacuugaaaaaguggca | 67 | 38285 |
| | | ccgagucggugcuuu | | |
| min-mod/ | 30 | C3-cuuauauccaacacuucgugguuuuagagcuaugcu-C3 | 36 | HPRT |
| mod | 33 | a*g*cauagcaaguuaaaauaaggcuaguccguuaucaacuugaaaaaguggc | 67 | 38285 |
| | | accgagucggugcu*u*u | | |
| med mod/ | 31 | c*u*u*auauccaacacuucgugguuuuagagcuau*g*c*u | 36 | HPRT |
| mod | 33 | a*g*cauagcaaguuaaaauaaggcuaguccguuaucaacuugaaaaaguggc | 67 | 38285 |
| | | accgagucggugcu*u*u | | |
| heavy | 32 | c*u*u*auauccaacacuucgugguuuuagagcuau*g*c*u | 36 | HPRT |
| mod/ | 33 | a*g*cauagcaaguuaaaauaaggcuaguccguuaucaacuugaaaaaguggc | 67 | 38285 |
| mod | | accgagucggugcu*u*u | | |

Oligonucleotide sequences are shown 5′-3′. Lowercase = RNA, Underline = 2′-O-methyl RNA, * = phosphorothioate, +a, +c, +t, +g = LNA; C3 = C3 spacer (propanediol modifier). Lengths of RNA oligonucleotides are indicated (bases).

HPRT38285 crRNA (unmodified, Alt-R (min-mod) modified, medium modified or heavy modified) was complexed to unmodified tracrRNA or Alt-R tracrRNA at a 1:1 molar ratio. The various crRNA:tracrRNA complexes were delivered into Jurkat cells via Neon electroporation (Thermo Fisher; program: 1600 V, 10 ms, 3 pulses) at a final concentration of 18 μM with 1 μg Cas9 mRNA. gDNA was isolated after 72 hours using QuickExtract (Epicentre) and HPRT region of interest amplified with KAPA HiFi Polymerase (Kapa Biosystems). Heteroduplexes were formed by heating amplicons to 95° C. and slowly cooling to room temperature. Heteroduplexes were digested with 2 units of T7EI (IDT Alt-R Genome Editing Kit) at 37° C. for 60 min. Digested samples were analyzed for total editing by visualization on the Fragment Analyzer (Advanced Analytical).

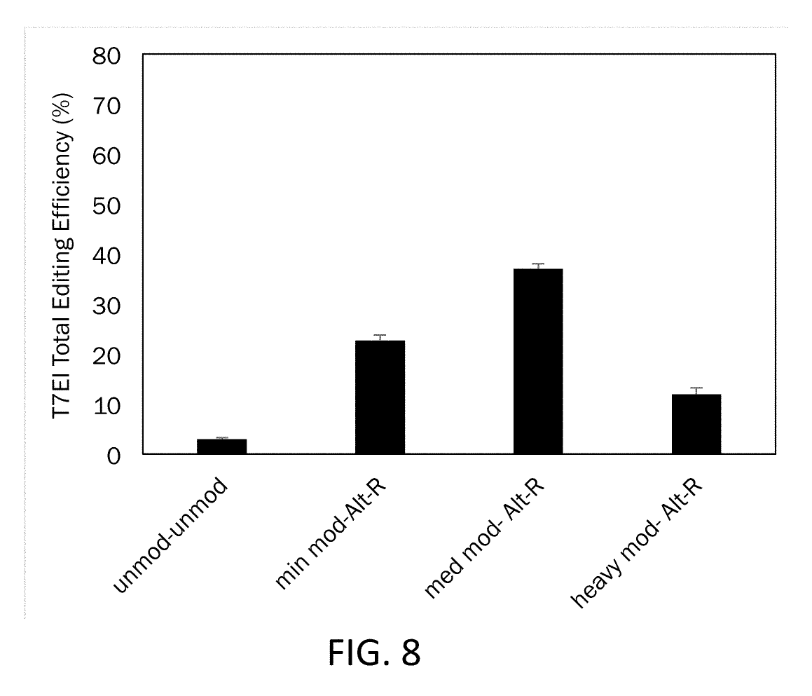

The data demonstrate that more highly modified crRNA can result in greater editing efficiency when the Cas9 protein is delivered as mRNA. FIG. 8 is a plot of the functional gene editing observed using the T7E1 assay in Jurkat cells using 2′-OMe and phosphorothioate modified crRNA. The medium modified crRNA (Med mod-Alt-R) had the highest activity and additional modifications are needed to protect the crRNA from nuclease attack until sufficient Cas9 protein is expressed from the transfected Cas9 mRNA.

### Example 5

Example 4 demonstrated that more highly chemically modified crRNA can show higher functional activity in mammalian gene editing when the Cas9 is delivered as mRNA instead of protein. The present example shows that LNA modified crRNA are effective and can direct CRISPR Cas editing in mammalian cells when the Cas9 is delivered as mRNA instead of protein.

A series of crRNAs (Table 7) targeting different the human HPRT1 gene were made. The crRNAs were either med-mod (SEQ ID No 0.31), LNA mod1 pattern (SEQ ID No. 3), LNA Mod2 pattern (SEQ ID No. 5). The crRNAs were duplexed with a modified tracrRNA (SEQ ID No 33).

TABLE 7
crRNA and tracrRNA pairs for use in in vivo biochemical studies of cleavage of
the HPRT1 target DNA by transfected Cas9 mRNA.

| cr/tracr | | | | |
|---|---|---|---|---|
| RNA | SEQ ID | crRNA Sequence | | Target |
| Pair | No. | tracrRNA Sequence | Length | Site |
| med | 31 | c*u*u*auauccaacacuucgugguuuuagagcuau*g*c*u | 36 | HPRT |
| mod/ | 33 | a*g*cauagcaaguuaaaauaaggcuaguccguuaucaacuugaaaaaguggc | 67 | |
| mod | | accgagucggugcu*u*u | | |
| LNA | 3 | c*u*uauauccaacacuucgugguuuuagagcuau+g*+c*u | 36 | HPRT |
| mod1/ | 33 | a*g*cauagcaaguuaaaauaaggcuaguccguuaucaacuugaaaaaguggc | 67 | 38285 |
| mod | | accgagucggugcu*u*u | | |
| LNA | 5 | c*u*uauauccaacacuucgugguuuuagagcuua+t+g*+c*u | 36 | HPRT |
| mod2/ | 33 | a*g*cauagcaaguuaaaauaaggcuaguccguuaucaacuugaaaaaguggc | 67 | 38285 |
| mod | | accgagucggugcu*u*u | | |

Oligonucleotide sequences are shown 5′-3′. Lowercase = RNA, Underline = 2′-O-methyl RNA, * = phosphorothioate, +a, +c, +t, +g = LNA; C3 = C3 spacer (propanediol modifier). Lengths of RNA oligonucleotides are indicated (bases).

HPRT38285 crRNA (medium mod, LNA-mod1 and LNA-mod2) was complexed to Alt-R tracrRNA at a 1:1 molar ratio. The various complexes were delivered into Jurkat cells via Neon electroporation (Thermo Fisher; program: 1600 V, 10 ms, 3 pulses) at a final concentration of 18 μM or 1.8 μM with 1 μg Cas9 mRNA. gDNA was isolated after 72 hours using QuickExtract (Epicentre) and HPRT region of interest amplified with KAPA HiFi Polymerase (Kapa Biosystems). Heteroduplexes were created by heating amplicons to 95° C. and slowly cooling to room temperature. Heteroduplexes were digested with 2 units of T7EI (IDT Alt-R Genome Editing Kit) at 37° C. for 60 min. Digested samples were analyzed for total editing by visualization on the Fragment Analyzer (Advanced Analytical).

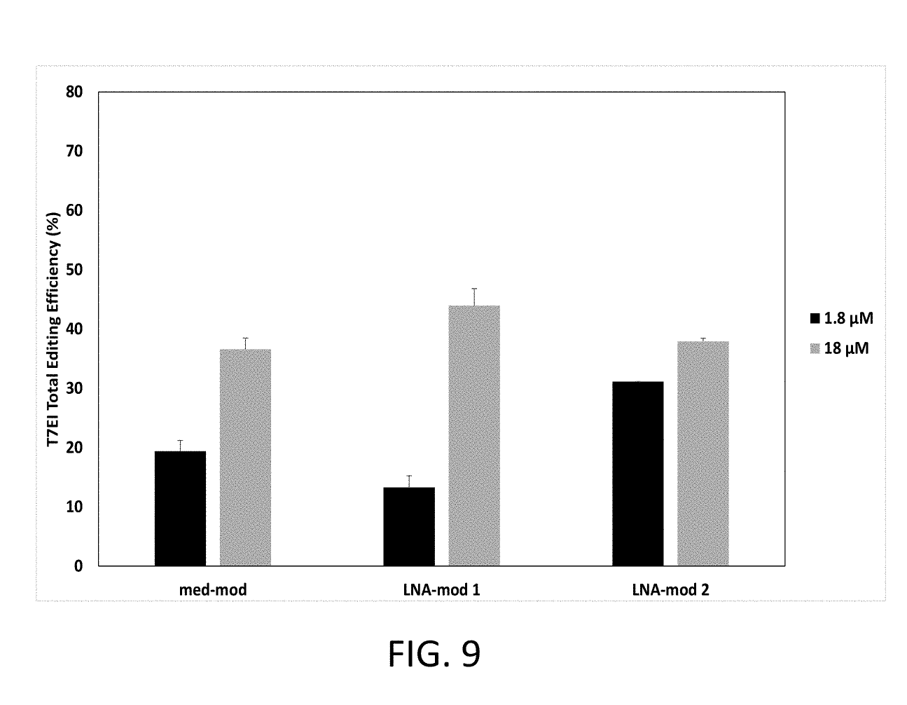

The data in FIG. 9 show that LNA modified crRNAs are capable of directing Cas9 cleavage and have similar improved activity as the medium modified crRNA. Further the data show that LNA modified crRNA can improve the editing efficiency.

### Example 6

### Use of Modified crRNAs with an SpCas9 Expression Plasmid in E. coli

A site on the human chromosome downstream of the VEGFA gene was cloned onto an E. coli plasmid and was used to study the ability to use chemically modified crRNAs to perform site-specific cleavage in E. coli cells. SpCas9 was expressed from a plasmid. Electroporation was used to deliver both the SpCas9 expression plasmid and the chemically-synthesized crRNAs.

The SpCas9 protein was expressed from a plasmid expression construct in this example, using a phage T7 promoter and standard E. coli translation elements. The nucleotide sequence of the plasmid expression construct is shown in SEQ ID NO:48.

Nucleotide sequence of pACYCDuet-1-EcCas9.
SEQ ID NO: 48
GGGGAATTGTGAGCGGATAACAATTCCCCTGTAGAAATAATTTTGTTTAA
CTTTAATAAGGAGATATACCATGGACAAAAAGTACTCTATTGGCCTGGAT
ATCGGGACCAACAGCGTCGGGTGGGCTGTTATCACCGACGAGTATAAAGT
ACCTTCGAAAAAGTTCAAGTGCTGGGCAACACCGATCGCCATTCAATCAA
AAAGAACTTGATTGGTGCGCTGTTGTTTGACTCCGGGGAAACCGCCGAGG
CGACTCGCCTTAAACGTACAGCACGTCGCCGGTACACTCGGCGTAAGAAT
CGCATTTGCTATTTGCAGGAAATCTTTAGCAACGAGATGGCAAAAGTCGA
TGACTCGTTTTTCCACCGCCTCGAGGAAAGCTTTCTGGTGGAGGAAGACA
AAAAGCATGAGCGTCACCCGATCTTCGGCAACATTGTCGATGAAGTAGCG
TATCATGAAAAATACCCAACCATTTACCACTTACGCAAAAAGCTGGTGGA
CAGCACTGACAAAGCTGATTTGCGCCTTATCTATTTAGCCCTGGCACATA
TGATTAAGTTTCGTGGTCACTTCCTGATCGAAGGAGACTTAAATCCCGAC
AACAGTGATGTTGATAAATTGTTTATTCAGCTTGTCCAAACTTACAATCA
ACTGTTCGAGGAAAACCCGATCAATGCCTCCGGTGTGGATGCAAAAGCCA
TTTTAAGTGCACGCCTTAGCAAGTCCCGTCGCTTAGAAAACCTTATCGCG
CAGCTGCCCGGCGAGAAAAAGAATGGTTTGTTTGGGAACCTTATTGCCTT
GAGCTTAGGCCTCACCCCGAATTTCAAAAGTAATTTCGATCTTGCAGAAG
ACGCCAAATTACAACTGTCGAAGGATACTTATGATGACGATCTCGATAAT
CTGTTAGCGCAGATTGGTGACCAATACGCCGATCTTTTTCTGGCGGCTAA
AAATCTGAGCGACGCCATCTTGCTTTCGGATATTCTCCGCGTTAACACCG
AAATCACGAAAGCGCCTCTTAGTGCCAGCATGATTAAACGTTATGATGAA
CACCACCAGGACCTGACCTTACTCAAAGCGTTGGTTCGCCAGCAACTGCC
AGAGAAGTACAAAGAAATCTTCTTTGATCAGTCAAAGAATGGTTATGCCG
GCTATATTGACGGGGGTGCAAGCCAAGAGGAATTCTACAAATTTATCAAG
CCTATTCTGGAGAAAATGGATGGCACCGAAGAGTTATTGGTGAAGCTTAA
CCGTGAAGACCTCCTGCGGAAACAGCGCACATTCGATAATGGTTCGATCC
CACACCAAATCCATTTGGGGGAGTTACACGCTATTTTGCGTCGCCAGGAA
GACTTTTACCCTTTCCTGAAGGATAACCGGGAGAAAATTGAGAAGATCCT
TACCTTTCGTATTCCGTATTACGTAGGCCCCTTAGCACGGGGTAATAGCC
GTTTCGCGTGGATGACACGGAAGTCGGAAGAGACGATCACCCCGTGGAAC
TTCGAAGAGGTAGTCGACAAGGGCGCATCAGCGCAGTCTTTTATTGAACG
TATGACGAATTTCGATAAAAACTTGCCCAATGAGAAGGTGCTTCCGAAAC
ATTCCTTGTTATATGAATATTTTACAGTTTACAACGAGCTGACCAAGGTT
AAATACGTGACGGAAGGAATGCGCAAGCCCGCTTTTCTTAGCGGTGAGCA
AAAAAAGGCGATCGTCGACCTGTTATTCAAAACGAATCGTAAGGTGACTG
TAAAGCAACTCAAAGAAGATTACTTCAAAAAGATTGAGTGCTTCGACAGC
GTCGAAATCTCTGGGGTAGAGGATCGGTTTAACGCAAGTTTAGGTACCTA
CCATGACCTGCTTAAAATCATTAAGGATAAAGACTTCTTAGATAATGAAG
AGAACGAAGATATTCTCGAGGACATCGTCTTGACGTTAACCTTATTTGAG
GATCGTGAAATGATTGAGGAACGCCTCAAAACTATGCCCACCTGTTCGAC
GATAAGGTGATGAAGCAGCTGAAACGTCGGCGCTACACAGGATGGGGCCG
CTTGAGTCGCAAACTTATTAACGGAATCCGTGACAAGCAATCCGGCAAAA
CGATTCTGGATTTCTTGAAGTCGGACGGATTTGCTAATCGCAACTTCATG
CAGTTGATCCATGATGACTCCCTGACTTTTAAAGAGGATATTCAAAAGGC
GCAGGTTAGTGGTCAAGGCGACAGCTTACACGAACACATCGCAAATTTGG
CTGGTTCGCCGGCCATTAAAAAGGGGATCCTCCAGACCGTGAAAGTTGTA
GATGAGCTTGTTAAGGTCATGGGTCGTCATAAGCCCGAAAACATCGTGAT
TGAAATGGCGCGGGAGAATCAAACGACCCAGAAAGGACAAAAGAATAGCC
GTGAACGGATGAAGCGGATCGAGGAAGGCATTAAAGAGCTGGGGTCTCAA
ATCTTGAAGGAACACCCTGTGGAGAACACTCAGCTCCAAAATGAAAAACT
TTACCTGTACTATTTGCAGAACGGACGCGATATGTACGTGGACCAAGAGT
TGGATATTAATCGGCTGAGTGACTACGACGTTGATCATATCGTCCCGCAG
AGCTTCCTCAAAGACGATTCTATTGACAATAAGGTACTGACGCGCTCTGA
TAAAAACCGTGGTAAGTCGGACAACGTGCCCTCCGAAGAGGTTGTGAAAA
AGATGAAAAATTATTGGCGCCAGCTTTTAAACGCGAAGCTGATCACACAA
CGTAAATTCGATAATTTGACCAAGGCTGAACGGGGTGGCCTAGCGAGTTA
GATAAGGCAGGATTTATTAAACGCCAGTTAGTGGAGACTCGTCAAATCAC
CAAACATGTCGCGCAGATTTTGGACAGCCGGATGAACACCAAGTACGATG
AAAATGACAAACTGATCCGTGAGGTGAAAGTCATTACTCTGAAGTCCAAA
TTAGTTAGTGATTTCCGGAAGGACTTTCAATTCTACAAAGTCCGTGAAAT
TAATAACTATCATCACGCACATGACGCGTACCTGAATGCAGTGGTTGGGA
CCGCCCTTATCAAGAAATATCCTAAGCTGGAGTCGGAGTTTGTCTATGGC
GACTATAAGGTATACGATGTTCGCAAAATGATTGCGAAATCTGAGCAGGA
GATCGGTAAGGCAACCGCAAAATATTTCTTTTACTCAAACATTATGAATT
TCTTTAAGACAGAAATCACTCTGGCCAACGGGGAGATTCGCAAACGTCCG
TTGATCGAAACAAACGGCGAGACTGGCGAAATTGTTTGGGACAAAGGGCG
TGATTTCGCGACGGTGCGCAAGGTACTGAGCATGCCTCAAGTCAATATTG
TTAAGAAAACCGAAGTGCAGACGGGCGGGTTTTCCAAGGAAAGCATCTTA
CCCAAACGTAATTCAGATAAACTTATTGCACGCAAAAAGGACTGGGATCC
GAAAAAGTATGGAGGCTTCGACAGTCCAACCGTAGCCTACTCTGTTCTCG
TTGTAGCGAAAGTAGAAAAGGGTAAATCCAAGAAACTGAAATCTGTCAAG
GAGTTGCTTGGAATCACCATTATGGAGCGTAGCTCCTTCGAGAAGAACCC
GATTGACTTTCTGGAAGCCAAAGGATATAAAGAGGTCAAGAAAGATCTTA
TCATTAAGCTGCCTAAGTATTCACTCTTCGAGCTGGAAAATGGTCGTAAA
CGCATGCTCGCTTCTGCCGGCGAGTTGCAGAAGGGCAATGAATTAGCACT
TCCATCAAAGTACGTTAACTTCCTGTATTTGGCCAGCCATTACGAGAAAC
TGAAGGGGTCTCCAGAGGACAACGAACAGAAACAATTATTTGTAGAGCAG
CACAAGCATTATCTTGATGAAATCATTGAGCAAATTTCCGAATTCAGTAA
ACGCGTAATCCTGGCCGATGCAAACCTCGACAAGGTGCTGAGCGCTTACA
ATAAGCATCGCGACAAACCTATCCGTGAGCAGGCTGAAAATATCATTCAC
CTGTTCACATTAACGAACCTGGGCGCTCCGGCCGCTTTTAAATATTTCGA
CACGACAATCGACCGTAAGCGCTATACCAGTACGAAAGAAGTGTTGGATG
CGACCCTTATTCACCAGTCAATTACAGGATTATATGAGACCCGTATCGAC
CTTAGCCAATTAGGTGGGGATTAAGAGCTCGGCGCGCCTGCAGGTCGACA
AGCTTGCGGCCGCATAATGCTTAAGTCGAACAGAAAGTAATCGTATTGTA
CACGGCCGCATAATCGAAATTAATACGACTCACTATAGGGGAATTGTGAG
CGGATAACAATTCCCCATCTTAGTATATTAGTTAAGTATAAGAAGGAGAT
ATACATATGGCAGATCTCAATTGGATATCGGCCGGCCACGCGATCGCTGA
CGTCGGTACCCTCGAGTCTGGTAAAGAAACCGCTGCTGCGAAATTTGAAC
GCCAGCACATGGACTCGTCTACTAGCGCAGCTTAATTAACCTAGGCTGCT
GCCACCGCTGAGCAATAACTAGCATAACCCCTTGGGGCCTCTAAACGGGT
CTTGAGGGGTTTTTTGCTGAAACCTCAGGCATTTGAGAAGCACACGGTCA
CACTGCTTCCGGTAGTCAATAAACCGGTAAACCAGCAATAGACATAAGCG
GCTATTTAACGACCCTGCCCTGAACCGACGACCGGGTCGAATTTGCTTTC
GAATTTCTGCCATTCATCCGCTTATTATCACTTATTCAGGCGTAGCACCA
GGCGTTTAAGGGCACCAATAACTGCCTTAAAAAAATTACGCCCCGCCCTG
CCACTCATCGCAGTACTGTTGTAATTCATTAAGCATTCTGCCGACATGGA
AGCCATCACAGACGGCATGATGAACCTGAATCGCCAGCGGCATCAGCACC
TTGTCGCCTTGCGTATAATATTTGCCCATAGTGAAAACGGGGGCGAAGAA
GTTGTCCATATTGGCCACGTTTAAATCAAAACTGGTGAAACTCACCCAGG
GATTGGCTGAGACGAAAAACATATTCTCAATAAACCCTTTAGGGAAATAG
GCCAGGTTTTCACCGTAACACGCCACATCTTGCGAATATATGTGTAGAAA
CTGCCGGAAATCGTCGTGGTATTCACTCCAGAGCGATGAAAACGTTTCAG
TTTGCTCATGGAAAACGGTGTAACAAGGGTGAACACTATCCCATATCACC
AGCTCACCGTCTTTCATTGCCATACGGAACTCCGGATGAGCATTCATCAG
GCGGGCAAGAATGTGAATAAAGGCCGGATAAAACTTGTGCTTATTTTTCT
TTACGGTCTTTAAAAAGGCCGTAATATCCAGCTGAACGGTCTGGTTATAG
GTACATTGAGCAACTGACTGAAATGCCTCAAAATGTTCTTTACGATGCCA
TTGGGATATATCAACGGTGGTATATCCAGTGATTTTTTTCTCCATTTTAG
CTTCCTTAGCTCCTGAAAATCTCGATAACTCAAAAAATACGCCCGTAGTG
ATCTTATTTCATTATGGTGAAAGTTGGAACCTCTTACGTGCCGATCAACG
TCTCATTTTCGCCAAAAGTTGGCCCAGGGCTTCCCGGTATCAACAGGGAC
ACCAGGATTTATTTATTCTGCGAAGTGATCTTCCGTCACAGGTATTTATT
CGGCGCAAAGTGCGTCGGGTGATGCTGCCAACTTACTGATTTAGTGTATG
ATGGTGTTTTTGAGGTGCTCCAGTGGCTTCTGTTTCTATCAGCTGTCCCT
CCTGTTCAGCTACTGACGGGGTGGTGCGTAACGGCAAAAGCACCGCCGGA
CATCAGCGCTAGCGGAGTGTATACTGGCTTACTATGTTGGCACTGATGAG
GGTGTCAGTGAAGTGCTTCATGTGGCAGGAGAAAAAAGGCTGCACCGGTG
CGTCAGCAGAATATGTGATACAGGATATATTCCGCTTCCTCGCTCACTGA
CTCGCTACGCTCGGTCGTTCGACTGCGGCGAGCGGAAATGGCTTACGAAC
GGGGCGGAGATTTCCTGGAAGATGCCAGGAAGATACTTAACAGGGAAGTG
AGAGGGCCGCGGCAAAGCCGTTTTTCCATAGGCTCCGCCCCCCTGACAAG
CATCACGAAATCTGACGCTCAAATCAGTGGTGGCGAAACCCGACAGGACT
ATAAAGATACCAGGCGTTTCCCCTGGCGGCTCCCTCGTGCGCTCTCTGTT
CCTGCCTTTCGGTTTACCGGTGTCATTCCGCTGTTATGGCCGCGTTTGTC
TCATTCCACGCCTGACACTCAGTTCCGGGTAGGCAGTTCGCTCCAAGCTG
GACTGTATGCACGAACCCCCCGTTCAGTCCGACCGCTGCGCCTTATCCGG
TAACTATCGTCTTGAGTCCAACCCGGAAAGACATGCAAAAGCACCACTGG
CAGCAGCCACTGGTAATTGATTTAGAGGAGTTAGTCTTGAAGTCATGCGC
CGGTTAAGGCTAAACTGAAAGGACAAGTTTTGGTGACTGCGCTCCTCCAA
GCCAGTTACCTCGGTTCAAAGAGTTGGTAGCTCAGAGAACCTTCGAAAAA
CCGCCCTGCAAGGCGGTTTTTTCGTTTTCAGAGCAAGAGATTACGCGCAG
ACCAAAACGATCTCAAGAAGATCATCTTATTAATCAGATAAAATATTTCT
AGATTTCAGTGCAATTTATCTCTTCAAATGTAGCACCTGAAGTCAGCCCC
ATACGATATAAGTTGTAATTCTCATGTTAGTCATGCCCCGCGCCCACCGG
AAGGAGCTGACTGGGTTGAAGGCTCTCAAGGGCATCGGTCGAGATCCCGG
TGCCTAATGAGTGAGCTAACTTACATTAATTGCGTTGCGCTCACTGCCCG
CTTTCCAGTCGGGAAACCTGTCGTGCCAGCTGCATTAATGAATCGGCCAA
CGCGCGGGGAGAGGCGGTTTGCGTATTGGGCGCCAGGGTGGTTTTTCTTT
TCACCAGTGAGACGGGCAACAGCTGATTGCCCTTCACCGCCTGGCCCTGA
GAGAGTTGCAGCAAGCGGTCCACGCTGGTTTGCCCCAGCAGGCGAAAATC
CTGTTTGATGGTGGTTAACGGCGGGATATAACATGAGCTGTCTTCGGTAT
CGTCGTATCCCACTACCGAGATGTCCGCACCAACGCGCAGCCCGGACTCG
GTAATGGCGCGCATTGCGCCCAGCGCCATCTGATCGTTGGCAACCAGCAT
CGCAGTGGGAACGATGCCCTCATTCAGCATTTGCATGGTTTGTTGAAAAC
CGGACATGGCACTCCAGTCGCCTTCCCGTTCCGCTATCGGCTGAATTTGA
TTGCGAGTGAGATATTTATGCCAGCCAGCCAGACGCAGACGCGCCGAGAC
AGAACTTAATGGGCCCGCTAACAGCGCGATTTGCTGGTGACCCAATGCGA
CCAGATGCTCCACGCCCAGTCGCGTACCGTCTTCATGGGAGAAAATAATA
CTGTTGATGGGTGTCTGGTCAGAGACATCAAGAAATAACGCCGGAACATT
AGTGCAGGCAGCTTCCACAGCAATGGCATCCTGGTCATCCAGCGGATAGT
TAATGATCAGCCCACTGACGCGTTGCGCGAGAAGATTGTGCACCGCCGCT
TTACAGGCTTCGACGCCGCTTCGTTCTACCATCGACACCACCACGCTGGC
ACCCAGTTGATCGGCGCGAGATTTAATCGCCGCGACAATTTGCGACGGCG
CGTGCAGGGCCAGACTGGAGGTGGCAACGCCAATCAGCAACGACTGTTTG
CCCGCCAGTTGTTGTGCCACGCGGTTGGGAATGTAATTCAGCTCCGCCAT
CGCCGCTTCCACTTTTTCCCGCGTTTTCGCAGAAACGTGGCTGGCCTGGT
TCACCACGCGGGAAACGGTCTGATAAGAGACACCGGCATACTCTGCGACA
TCGTATAACGTTACTGGTTTCACATTCACCACCCTGAATTGACTCTCTTC
CGGGCGCTATCATGCCATACCGCGAAAGGTTTTGCGCCATTCGATGGTGT
CCGGGATCTCGACGCTCTCCCTTATGCGACTCCTGCATTAGGAAATTAAT
ACGACTCACTATA

The amino acid sequence of the SpCas9 protein produced from this plasmid expression construct is shown in SEQ ID NO:35.

Amino acid sequence of SpCas9 protein.
SEQ ID NO. 35
MDKKYSIGLDIGTNSVGWAVITDEYKVPSKKEKVLGNTDRHSIKKNLIGA
LLEDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

The SpCas9 crRNAs were duplexed to modified tracrRNA (SEQ ID No. 33) at a 1:1 ratio (final concentration 100 μM) by heating to 95° C. for 5 minutes and then allowing the heteroduplex to cool to room temperature. The crRNA:tracrRNA complexes and SpCas9 plasmid were mixed in TE (60 femtomoles SpCas9 plasmid with 200 pmoles RNA complex in 5 μL volume, for a single transformation), and added directly to 20 μL of competent E. coli cells. A bacterial strain where survival is linked to successful cleavage by Cas9 was made competent by growing cells to mid-log phase, washing 3 times in ice cold 10% glycerol, and final suspension in 1:100^{th} volume 10% glycerol. Electroporations were performed by adding the 25 μL transformation mixture to a pre-chilled 0.1 cm electroporation cuvette and pulsing 1.8 kV exponential decay. Following electroporation, 980 μL of SOB medium was added to the electroporation cuvette with mixing and the resulting cell suspension was transferred to a sterile 15 mL culture tube. Cells were incubated with shaking (250 rpm) at 37° C. for 1 hour and then plated on selective media to assess survival.

This example demonstrates that chemically-modified synthetic crRNAs can be used with Cas9 for gene editing in bacteria. However, high efficiency is only seen using RNAs that have been more extensively modified with exonuclease-blocking PS internucleotide linkages. Synthetic crRNAs lacking PS linkages that work well in mammalian cells do not work in bacterial cells (Table 8).

TABLE 8
Chemically-modified crRNAs compatible with Cas9 function in bacteria.

| cr/tracr | | | % | % | |
|---|---|---|---|---|---|
| RNA | SEQ ID | crRNA Sequence | Cleavage | Cleavage | Target |
| Pair | No. | tracrRNA Sequence | Human | Bacteria | Site |
| unmod/ | 45 | ggugagugagugugugcgugguuuuagagcuaugcu | 55 | 0 | VEGFA3 |
| mod | 33 | a*g*cauagcaaguuaaaauaaggcuaguccguuaucaa | | | |
| | | cuugaaaaaguggcaccgagucggugcu*u*u | | | |
| minmod/ | 46 | C3- | 60 | 0 | VEGFA3 |
| mod | | ggugagugagugugugcgugguuuuagagcuaugcu-C3 | | | |
| | 33 | a*g*cauagcaaguuaaaauaaggcuaguccguuaucaa | | | |
| | | cuugaaaaaguggcaccgagucggugcu*u*u | | | |
| 6PS/ | 47 | g*g*u*g*a*g*ugagugugugcgugguuuuagagc*u | 59 | 60 | VEGFA3 |
| mod | | *a*u*g*c*u | | | |
| | 33 | a*g*cauagcaaguuaaaauaaggcuaguccguuaucaa | | | |
| | | cuugaaaaaguggcaccgagucggugcu*u*u | | | |

Oligonucleotide sequences are shown 5′-3′. Lowercase = RNA; Underlined lowercase = 2′-O-methyl RNA; C3 = C3 spacer (propanediol modifier); * = phosphorothioate internucleotide linkage. The relative functional activity in human cells is indicated by the % cleavage in a T7EI heteroduplex assay, and in bacteria is indicated by % survival in a Cas9 reporter strain.

Sequences presented in this application are presented below.

| | SEQ | |
|---|---|---|
| | ID | |
| Oligonucleotide | No. | Sequence (5′-3′) |
| unmod--crRNA | 1 | cuuauauccaacacuucgugguuuuagagcuaugcu |
| HPRT 38285 AS | 2 | C3-cuuauauccaacacuucgugguuuuagagcuaugcu-C3 |
| Alt-R | | |
| LNA-Mod1 | 3 | c*u*uauauccaacacuucgugguuuuagagcuau+g*+c*u |
| Heavy | 4 | c*u*u*auauccaacacuucgugguuuuagagcuau*g*c*u |
| LNA mod2 | 5 | c*u*uauauccaacacuucgugguuuuagagcua+t+g*+c* |
| | | u |
| LNA mod3 | 6 | c*u*uauauccaacacuucgugguuuuaga+g+cuau+g*+c |
| | | *u |
| LNA mod4 | 7 | c*u*uauauccaacacuucgugguuuuaga+g+cuau+g*+c |
| | | *u |
| LNA mod2 HPRT | 8 | c*u*uauauccaacacuucgugguuuuagagcua+t+g*+c* |
| 38285 | | u |
| HPRT 38285 AS | 9 | c*u*u*auauccaacacuucgugguuuuagagcuagaaauag |
| chem mod sgRNA | | caaguuaaaauaaggcuaguccguuaucaacuugaaaaagug |
| | | gcaccgagucggugcu*u*u*u |
| MYC-S-459 | 10 | C3-gaggcuauucugcccauuugguuuuagagcuaugcu-C3 |
| MYC-S-459 LNA | 11 | g*a*ggcuauucugcccauuugguuuuagagcuau+g*+c*u |
| Mod | | |
| MYC-S-459 LNA | 12 | g*a*ggcuauucugcccauuugguuuuagagcua+t+g*+c* |
| Mod2 | | u |
| MYC-S-459 | 13 | g*a*ggcuauucugcccauuugguuuuagagcuagaaauagc |
| sgRNA mod1 | | aaguuaaaauaaggcuaguccguuaucaacuugaaaaagugg |
| | | caccgagucggugcu*u*u*u |
| HAMP-S-253 | 14 | C3-uggcacugagcucccagaucguuuuagagcuaugcu-C3 |
| HAMP-S-253 LNA | 15 | u*g*gcacugagcucccagaucguuuuagagcuau+g*+c*u |
| Mod | | |
| HAMP-S-253 LNA | 16 | u*g*gcacugagcucccagaucguuuuagagcua+t+g*+c* |
| Mod2 | | u |
| HAMP-S-253 | 17 | u*g*gcacugagcucccagaucguuuuagagcuagaaauagc |
| sgRNA mod1 | | aaguuaaaauaaggcuaguccguuaucaacuugaaaaagugg |
| | | caccgagucggugcu*u*u*u |
| APOC3-S-2929 | 18 | C3-aggacaaguucucugaguucguuuuagagcuaugcu-C3 |
| APOC3-S-2929 | 19 | a*g*gacaaguucucugaguucguuuuagagcuau+g*+c*u |
| LNA Mod | | |
| APOC3-S-2929 | 20 | a*g*gacaaguucucugaguucguuuuagagca+t+g*+c*u |
| LNA Mod2 | | |
| APOC3-S-2929 | 21 | a*g*gacaaguucucugaguucguuuuagagcuagaaauagc |
| sgRNA mod1 | | aaguuaaaauaaggcuaguccguuaucaacuugaaaaagugg |
| | | caccgagucggugcu*u*u*u |
| SERPINA1-AS- | 22 | C3-ccccuccaaccuggaauuccguuuuagagcuaugcu-C3 |
| 130 | | |
| SERPINA1-AS- | 23 | c*c*ccuccaaccuggaauuccguuuuagagcuau+g*+c*u |
| 130 LNA Mod | | |
| SERPINA1-AS- | 24 | c*c*ccuccaaccuggaauuccguuuuagagcua+t+g*+c* |
| 130 LNA Mod2 | | u |
| SERPINA1-AS- | 25 | c*c*ccuccaaccuggaauuccguuuuagagcuagaaauagc |
| 130 sgRNA mod1 | | aaguuaaaauaaggcuaguccguuaucaacuugaaaaagugg |
| | | caccgagucggugcu*u*u*u |
| STAT3-AS-39988 | 26 | C3-gcugcuguagcugauuccauguuuuagagcuaugcu-C3 |
| STAT3-AS-39988 | 27 | g*c*ugcuguagcugauuccauguuuuagagcuau+g*+c*u |
| LNA Mod | | |
| STAT3-AS-39988 | 28 | g*c*ugcuguagcugauuccauguuuuagagcua+t+g*+c* |
| LNA Mod2 | | u |
| STAT3-AS-39988 | 29 | g*c*ugcuguagcugauuccauguuuuagagcuagaaauagc |
| sgRNA mod1 | | aaguuaaaauaaggcuaguccguuaucaacuugaaaaagugg |
| | | caccgagucggugcu*u*u*u |
| Min-Mod | 30 | C3-cuuauauccaacacuucgugguuuuagagcuaugcu-C3 |
| Med-Mod | 31 | c*u*u*auauccaacacuucgugguuuuagagcuau*g*c*u |
| Heavy | 32 | c*u*u*auauccaacacuucgugguuuuagagcuau*g*c*u |
| Alt-R tracr | 33 | a*g*cauagcaaguuaaaauaaggcuaguccguuaucaacuu |
| Mod | | gaaaaaguggcaccgagucggugcu*u*u |
| tracr unmod- | 34 | agcauagcaaguuaaaauaaggcuaguccguuaucaacuuga |
| truncated | | aaaaguggcaccgagucggugcuuu |
| Cas9 protein | 35 | MDKKYSIGLDIGINSVGWAVITDEYKVPSKKFKVLGNIDRHS |
| amino acid | | IKKNLIGALLFDSGETAEATRLKRTARRRYTRRKNRICYLQE |
| sequence | | IFSNEMAKVDDSFFHRLEESFLVEEDKKHERHPIFGNIVDEV |
| | | AYHEKYPTIYHLRKKLVDSTDKADLRLIYLALAHMIKFRGHF |
| | | LIEGDLNPDNSDVDKLFIQLVQTYNQLFEENPINASGVDAKA |
| | | ILSARLSKSRRLENLIAQLPGEKKNGLFGNLIALSLGLTPNF |
| | | KSNFDLAEDAKLQLSKDTYDDDLDNLLAQIGDQYADLFLAAK |
| | | NLSDAILLSDILRVNTEITKAPLSASMIKRYDEHHQDLTLLK |
| | | ALVRQQLPEKYKEIFFDQSKNGYAGYIDGGASQEEFYKFIKP |
| | | ILEKMDGTEELLVKLNREDLLRKQRTFDNGSIPHQIHLGELH |
| | | AILRRQEDFYPFLKDNREKIEKILTFRIPYYVGPLARGNSRF |
| | | AWMTRKSEETITPWNFEEVVDKGASAQSFIERMINFDKNLPN |
| | | EKVLPKHSLLYEYFTVYNELTKVKYVTEGMRKPAFLSGEQKK |
| | | AIVDLLFKINRKVIVKQLKEDYFKKIECFDSVEISGVEDRFN |
| | | ASLGTYHDLLKIIKDKDFLDNEENEDILEDIVLILTLFEDRE |
| | | MIEERLKTYAHLFDDKVMKQLKRRRYTGWGRLSRKLINGIRD |
| | | KQSGKTILDFLKSDGFANRNFMQLIHDDSLTFKEDIQKAQVS |
| | | GQGDSLHEHIANLAGSPAIKKGILQTVKVVDELVKVMGRHKP |
| | | ENIVIEMARENQTTQKGQKNSRERMKRIEEGIKELGSQILKE |
| | | HPVENTQLQNEKLYLYYLQNGRDMYVDQELDINRLSDYDVDH |
| | | IVPQSFLKDDSIDNKVLTRSDKNRGKSDNVPSEEVVKKMKNY |
| | | WRQLLNAKLITQRKFDNLTKAERGGLSELDKAGFIKRQLVET |
| | | RQIIKHVAQILDSRMNIKYDENDKLIREVKVITLKSKLVSDF |
| | | RKDFQFYKVREINNYHHAHDAYLNAVVGTALIKKYPKLESEF |
| | | VYGDYKVYDVRKMIAKSEQEIGKATAKYFFYSNIMNFFKTEI |
| | | TLANGEIRKRPLIETNGETGEIVWDKGRDFATVRKVLSMPQV |
| | | NIVKKTEVQTGGFSKESILPKRNSDKLIARKKDWDPKKYGGF |
| | | DSPTVAYSVLVVAKVEKGKSKKLKSVKELLGITIMERSSFEK |
| | | NPIDFLEAKGYKEVKKDLIIKLPKYSLFELENGRKRMLASAG |
| | | ELQKGNELALPSKYVNFLYLASHYEKLKGSPEDNEQKQLFVE |
| | | QHKHYLDEIIEQISEFSKRVILADANLDKVLSAYNKHRDKPI |
| | | REQAENIIHLFTLINLGAPAAFKYFDTTIDRKRYISTKEVLD |
| | | ATLIHQSITGLYETRIDLSQLGGD |
| WT-to HPRT | 36 | cuuauauccaacacuucgugguuuuagagcuaugcuguuuug |
| Truncated-to | 37 | cuuauauccaacacuucgugguuuuagagcuaugcu |
| HPRT | | |
| WT | 38 | guuggaaccauucaaaacagcauagcaaguuaaaauaaggcu |
| | | aguccguuaucaacuugaaaaaguggcaccgagucggugcuu |
| | | uuuuu |
| sgRNA | 39 | cuuauauccaacacuucgugguuuuagagcuagaaauagcaa |
| | | guuaaaauaaggcuaguccguuaucaacuugaaaaaguggca |
| | | ccgagucggugcuuu |
| | 40 | c*u*u*auauccaacacuucgugguuuuagagcuau*g*c*u |
| LNA-Mod1- | 41 | guuuuagagcuau+g*+c+u |
| tracrRNA | | |
| binding | | |
| LNA mod2- | 42 | guuuuagagcuua+t+g*+c*u |
| tracrRNA | | |
| binding | | |
| LNA mod3- | 43 | guuuuaga+g+cuau+g*+c*u |
| tracrRNA | | |
| binding | | |
| LNA mod4- | 44 | guuuuaga+g+cuau+g*+c*u |
| tracrRNA | | |
| binding | | |
| VEGFA3-unmod | 45 | ggugagugagugugugcgugguuuuagagcuaugcu |
| VEGFA3-minmod | 46 | C3-ggugagugagugugugcgugguuuuagagcuaugcu-C3 |
| VEGFA3-6PS | 47 | g*g*u*g*a*g*ugagugugugcgugguuuuagagc*u*a*u |
| | | *g*c*u |
| pACYCDuet-1- | 48 | GGGGAATTGTGAGCGGATAACAATTCCCCTGTAGAAATAATT |
| EcCas9 DNA | | TTGTTTAACTTTAATAAGGAGATATACCATGGACAAAAAGTA |
| | | CTCTATTGGCCTGGATATCGGGACCAACAGCGTCGGGTGGGC |
| | | TGTTATCACCGACGAGTATAAAGTACCTTCGAAAAAGTTCAA |
| | | AGTGCTGGGCAACACCGATCGCCATTCAATCAAAAAGAACTT |
| | | GATTGGTGCGCTGTTGTTTGACTCCGGGGAAACCGCCGAGGC |
| | | GACTCGCCTTAAACGTACAGCACGTCGCCGGTACACTCGGCG |
| | | TAAGAATCGCATTTGCTATTTGCAGGAAATCTTTAGCAACGA |
| | | GATGGCAAAAGTCGATGACTCGTTTTTCCACCGCCTCGAGGA |
| | | AAGCTTTCTGGTGGAGGAAGACAAAAAGCATGAGCGTCACCC |
| | | GATCTTCGGCAACATTGTCGATGAAGTAGCGTATCATGAAAA |
| | | ATACCCAACCATTTACCACTTACGCAAAAAGCTGGTGGACAG |
| | | CACTGACAAAGCTGATTTGCGCCTTATCTATTTAGCCCTGGC |
| | | ACATATGATTAAGTTTCGTGGTCACTTCCTGATCGAAGGAGA |
| | | CTTAAATCCCGACAACAGTGATGTTGATAAATTGTTTATTCA |
| | | GCTTGTCCAAACTTACAATCAACTGTTCGAGGAAAACCCGAT |
| | | CAATGCCTCCGGTGTGGATGCAAAAGCCATTTTAAGTGCACG |
| | | CCTTAGCAAGTCCCGTCGCTTAGAAAACCTTATCGCGCAGCT |
| | | GCCCGGCGAGAAAAAGAATGGTTTGTTTGGGAACCTTATTGC |
| | | CTTGAGCTTAGGCCTCACCCCGAATTTCAAAAGTAATTTCGA |
| | | TCTTGCAGAAGACGCCAAATTACAACTGTCGAAGGATACTTA |
| | | TGATGACGATCTCGATAATCTGTTAGCGCAGATTGGTGACCA |
| | | ATACGCCGATCTTTTTCTGGCGGCTAAAAATCTGAGCGACGC |
| | | CATCTTGCTTTCGGATATTCTCCGCGTTAACACCGAAATCAC |
| | | GAAAGCGCCTCTTAGTGCCAGCATGATTAAACGTTATGATGA |
| | | ACACCACCAGGACCTGACCTTACTCAAAGCGTTGGTTCGCCA |
| | | GCAACTGCCAGAGAAGTACAAAGAAATCTTCTTTGATCAGTC |
| | | AAAGAATGGTTATGCCGGCTATATTGACGGGGGTGCAAGCCA |
| | | AGAGGAATTCTACAAATTTATCAAGCCTATTCTGGAGAAAAT |
| | | GGATGGCACCGAAGAGTTATTGGTGAAGCTTAACCGTGAAGA |
| | | CCTCCTGCGGAAACAGCGCACATTCGATAATGGTTCGATCCC |
| | | ACACCAAATCCATTTGGGGGAGTTACACGCTATTTTGCGTCG |
| | | CCAGGAAGACTTTTACCCTTTCCTGAAGGATAACCGGGAGAA |
| | | AATTGAGAAGATCCTTACCTTTCGTATTCCGTATTACGTAGG |
| | | CCCCTTAGCACGGGGTAATAGCCGTTTCGCGTGGATGACACG |
| | | GAAGTCGGAAGAGACGATCACCCCGTGGAACTTCGAAGAGGT |
| | | AGTCGACAAGGGCGCATCAGCGCAGTCTTTTATTGAACGTAT |
| | | GACGAATTTCGATAAAAACTTGCCCAATGAGAAGGTGCTTCC |
| | | GAAACATTCCTTGTTATATGAATATTTTACAGTTTACAACGA |
| | | GCTGACCAAGGTTAAATACGTGACGGAAGGAATGCGCAAGCC |
| | | CGCTTTTCTTAGCGGTGAGCAAAAAAAGGCGATCGTCGACCT |
| | | GTTATTCAAAACGAATCGTAAGGTGACTGTAAAGCAACTCAA |
| | | AGAAGATTACTTCAAAAAGATTGAGTGCTTCGACAGCGTCGA |
| | | AATCTCTGGGGTAGAGGATCGGTTTAACGCAAGTTTAGGTAC |
| | | CTACCATGACCTGCTTAAAATCATTAAGGATAAAGACTTCTT |
| | | AGATAATGAAGAGAACGAAGATATTCTCGAGGACATCGTCTT |
| | | GACGTTAACCTTATTTGAGGATCGTGAAATGATTGAGGAACG |
| | | CCTCAAAACTTATGCCCACCTGTTCGACGATAAGGTGATGAA |
| | | GCAGCTGAAACGTCGGCGCTACACAGGATGGGGCCGCTTGAG |
| | | TCGCAAACTTATTAACGGAATCCGTGACAAGCAATCCGGCAA |
| | | AACGATTCTGGATTTCTTGAAGTCGGACGGATTTGCTAATCG |
| | | CAACTTCATGCAGTTGATCCATGATGACTCCCTGACTTTTAA |
| | | AGAGGATATTCAAAAGGCGCAGGTTAGTGGTCAAGGCGACAG |
| | | CTTACACGAACACATCGCAAATTTGGCTGGTTCGCCGGCCAT |
| | | TAAAAAGGGGATCCTCCAGACCGTGAAAGTTGTAGATGAGCT |
| | | TGTTAAGGTCATGGGTCGTCATAAGCCCGAAAACATCGTGAT |
| | | TGAAATGGCGCGGGAGAATCAAACGACCCAGAAAGGACAAAA |
| | | GAATAGCCGTGAACGGATGAAGCGGATCGAGGAAGGCATTAA |
| | | AGAGCTGGGGTCTCAAATCTTGAAGGAACACCCTGTGGAGAA |
| | | CACTCAGCTCCAAAATGAAAAACTTTACCTGTACTATTTGCA |
| | | GAACGGACGCGATATGTACGTGGACCAAGAGTTGGATATTAA |
| | | TCGGCTGAGTGACTACGACGTTGATCATATCGTCCCGCAGAG |
| | | CTTCCTCAAAGACGATTCTATTGACAATAAGGTACTGACGCG |
| | | CTCTGATAAAAACCGTGGTAAGTCGGACAACGTGCCCTCCGA |
| | | AGAGGTTGTGAAAAAGATGAAAAATTATTGGCGCCAGCTTTT |
| | | AAACGCGAAGCTGATCACACAACGTAAATTCGATAATTTGAC |
| | | CAAGGCTGAACGGGGTGGCCTGAGCGAGTTAGATAAGGCAGG |
| | | ATTTATTAAACGCCAGTTAGTGGAGACTCGTCAAATCACCAA |
| | | ACATGTCGCGCAGATTTTGGACAGCCGGATGAACACCAAGTA |
| | | CGATGAAAATGACAAACTGATCCGTGAGGTGAAAGTCATTAC |
| | | TCTGAAGTCCAAATTAGTTAGTGATTTCCGGAAGGACTTTCA |
| | | ATTCTACAAAGTCCGTGAAATTAATAACTATCATCACGCACA |
| | | TGACGCGTACCTGAATGCAGTGGTTGGGACCGCCCTTATCAA |
| | | GAAATATCCTAAGCTGGAGTCGGAGTTTGTCTATGGCGACTA |
| | | TAAGGTATACGATGTTCGCAAAATGATTGCGAAATCTGAGCA |
| | | GGAGATCGGTAAGGCAACCGCAAAATATTTCTTTTACTCAAA |
| | | CATTATGAATTTCTTTAAGACAGAAATCACTCTGGCCAACGG |
| | | GGAGATTCGCAAACGTCCGTTGATCGAAACAAACGGCGAGAC |
| | | TGGCGAAATTGTTTGGGACAAAGGGCGTGATTTCGCGACGGT |
| | | GCGCAAGGTACTGAGCATGCCTCAAGTCAATATTGTTAAGAA |
| | | AACCGAAGTGCAGACGGGCGGGTTTTCCAAGGAAAGCATCTT |
| | | ACCCAAACGTAATTCAGATAAACTTATTGCACGCAAAAAGGA |
| | | CTGGGATCCGAAAAAGTATGGAGGCTTCGACAGTCCAACCGT |
| | | AGCCTACTCTGTTCTCGTTGTAGCGAAAGTAGAAAAGGGTAA |
| | | ATCCAAGAAACTGAAATCTGTCAAGGAGTTGCTTGGAATCAC |
| | | CATTATGGAGCGTAGCTCCTTCGAGAAGAACCCGATTGACTT |
| | | TCTGGAAGCCAAAGGATATAAAGAGGTCAAGAAAGATCTTAT |
| | | CATTAAGCTGCCTAAGTATTCACTCTTCGAGCTGGAAAATGG |
| | | TCGTAAACGCATGCTCGCTTCTGCCGGCGAGTTGCAGAAGGG |
| | | CAATGAATTAGCACTTCCATCAAAGTACGTTAACTTCCTGTA |
| | | TTTGGCCAGCCATTACGAGAAACTGAAGGGGTCTCCAGAGGA |
| | | CAACGAACAGAAACAATTATTTGTAGAGCAGCACAAGCATTA |
| | | TCTTGATGAAATCATTGAGCAAATTTCCGAATTCAGTAAACG |
| | | CGTAATCCTGGCCGATGCAAACCTCGACAAGGTGCTGAGCGC |
| | | TTACAATAAGCATCGCGACAAACCTATCCGTGAGCAGGCTGA |
| | | AAATATCATTCACCTGTTCACATTAACGAACCTGGGCGCTCC |
| | | GGCCGCTTTTAAATATTTCGACACGACAATCGACCGTAAGCG |
| | | CTATACCAGTACGAAAGAAGTGTTGGATGCGACCCTTATTCA |
| | | CCAGTCAATTACAGGATTATATGAGACCCGTATCGACCTTAG |
| | | CCAATTAGGTGGGGATTAAGAGCTCGGCGCGCCTGCAGGTCG |
| | | ACAAGCTTGCGGCCGCATAATGCTTAAGTCGAACAGAAAGTA |
| | | ATCGTATTGTACACGGCCGCATAATCGAAATTAATACGACTC |
| | | ACTATAGGGGAATTGTGAGCGGATAACAATTCCCCATCTTAG |
| | | TATATTAGTTAAGTATAAGAAGGAGATATACATATGGCAGAT |
| | | CTCAATTGGATATCGGCCGGCCACGCGATCGCTGACGTCGGT |
| | | ACCCTCGAGTCTGGTAAAGAAACCGCTGCTGCGAAATTTGAA |
| | | CGCCAGCACATGGACTCGTCTACTAGCGCAGCTTAATTAACC |
| | | TAGGCTGCTGCCACCGCTGAGCAATAACTAGCATAACCCCTT |
| | | GGGGCCTCTAAACGGGTCTTGAGGGGTTTTTTGCTGAAACCT |
| | | CAGGCATTTGAGAAGCACACGGTCACACTGCTTCCGGTAGTC |
| | | AATAAACCGGTAAACCAGCAATAGACATAAGCGGCTATTTAA |
| | | CGACCCTGCCCTGAACCGACGACCGGGTCGAATTTGCTTTCG |
| | | AATTTCTGCCATTCATCCGCTTATTATCACTTATTCAGGCGT |
| | | AGCACCAGGCGTTTAAGGGCACCAATAACTGCCTTAAAAAAA |
| | | TTACGCCCCGCCCTGCCACTCATCGCAGTACTGTTGTAATTC |
| | | ATTAAGCATTCTGCCGACATGGAAGCCATCACAGACGGCATG |
| | | ATGAACCTGAATCGCCAGCGGCATCAGCACCTTGTCGCCTTG |
| | | CGTATAATATTTGCCCATAGTGAAAACGGGGGCGAAGAAGTT |
| | | GTCCATATTGGCCACGTTTAAATCAAAACTGGTGAAACTCAC |
| | | CCAGGGATTGGCTGAGACGAAAAACATATTCTCAATAAACCC |
| | | TTTAGGGAAATAGGCCAGGTTTTCACCGTAACACGCCACATC |
| | | TTGCGAATATATGTGTAGAAACTGCCGGAAATCGTCGTGGTA |
| | | TTCACTCCAGAGCGATGAAAACGTTTCAGTTTGCTCATGGAA |
| | | AACGGTGTAACAAGGGTGAACACTATCCCATATCACCAGCTC |
| | | ACCGTCTTTCATTGCCATACGGAACTCCGGATGAGCATTCAT |
| | | CAGGCGGGCAAGAATGTGAATAAAGGCCGGATAAAACTTGTG |
| | | CTTATTTTTCTTTACGGTCTTTAAAAAGGCCGTAATATCCAG |
| | | CTGAACGGTCTGGTTATAGGTACATTGAGCAACTGACTGAAA |
| | | TGCCTCAAAATGTTCTTTACGATGCCATTGGGATATATCAAC |
| | | GGTGGTATATCCAGTGATTTTTTTCTCCATTTTAGCTTCCTT |
| | | AGCTCCTGAAAATCTCGATAACTCAAAAAATACGCCCGGTAG |
| | | TGATCTTATTTCATTATGGTGAAAGTTGGAACCTCTTACGTG |
| | | CCGATCAACGTCTCATTTTCGCCAAAAGTTGGCCCAGGGCTT |
| | | CCCGGTATCAACAGGGACACCAGGATTTATTTATTCTGCGAA |
| | | GTGATCTTCCGTCACAGGTATTTATTCGGCGCAAAGTGCGTC |
| | | GGGTGATGCTGCCAACTTACTGATTTAGTGTATGATGGTGTT |
| | | TTTGAGGTGCTCCAGTGGCTTCTGTTTCTATCAGCTGTCCCT |
| | | CCTGTTCAGCTACTGACGGGGTGGTGCGTAACGGCAAAAGCA |
| | | CCGCCGGACATCAGCGCTAGCGGAGTGTATACTGGCTTACTA |
| | | TGTTGGCACTGATGAGGGTGTCAGTGAAGTGCTTCATGTGGC |
| | | AGGAGAAAAAAGGCTGCACCGGTGCGTCAGCAGAATATGTGA |
| | | TACAGGATATATTCCGCTTCCTCGCTCACTGACTCGCTACGC |
| | | TCGGTCGTTCGACTGCGGCGAGCGGAAATGGCTTACGAACGG |
| | | GGCGGAGATTTCCTGGAAGATGCCAGGAAGATACTTAACAGG |
| | | GAAGTGAGAGGGCCGCGGCAAAGCCGTTTTTCCATAGGCTCC |
| | | GCCCCCCTGACAAGCATCACGAAATCTGACGCTCAAATCAGT |
| | | GGTGGCGAAACCCGACAGGACTATAAAGATACCAGGCGTTTC |
| | | CCCTGGCGGCTCCCTCGTGCGCTCTCCTGTTCCTGCCTTTCG |
| | | GTTTACCGGTGTCATTCCGCTGTTATGGCCGCGTTTGTCTCA |
| | | TTCCACGCCTGACACTCAGTTCCGGGTAGGCAGTTCGCTCCA |
| | | AGCTGGACTGTATGCACGAACCCCCCGTTCAGTCCGACCGCT |
| | | GCGCCTTATCCGGTAACTATCGTCTTGAGTCCAACCCGGAAA |
| | | GACATGCAAAAGCACCACTGGCAGCAGCCACTGGTAATTGAT |
| | | TTAGAGGAGTTAGTCTTGAAGTCATGCGCCGGTTAAGGCTAA |
| | | ACTGAAAGGACAAGTTTTGGTGACTGCGCTCCTCCAAGCCAG |
| | | TTACCTCGGTTCAAAGAGTTGGTAGCTCAGAGAACCTTCGAA |
| | | AAACCGCCCTGCAAGGCGGTTTTTTCGTTTTCAGAGCAAGAG |
| | | ATTACGCGCAGACCAAAACGATCTCAAGAAGATCATCTTATT |
| | | AATCAGATAAAATATTTCTAGATTTCAGTGCAATTTATCTCT |
| | | TCAAATGTAGCACCTGAAGTCAGCCCCATACGATATAAGTTG |
| | | TAATTCTCATGTTAGTCATGCCCCGCGCCCACCGGAAGGAGC |
| | | TGACTGGGTTGAAGGCTCTCAAGGGCATCGGTCGAGATCCCG |
| | | GTGCCTAATGAGTGAGCTAACTTACATTAATTGCGTTGCGCT |
| | | CACTGCCCGCTTTCCAGTCGGGAAACCTGTCGTGCCAGCTGC |
| | | ATTAATGAATCGGCCAACGCGCGGGGAGAGGCGGTTTGCGTA |
| | | TTGGGCGCCAGGGTGGTTTTTCTTTTCACCAGTGAGACGGGC |
| | | AACAGCTGATTGCCCTTCACCGCCTGGCCCTGAGAGAGTTGC |
| | | AGCAAGCGGTCCACGCTGGTTTGCCCCAGCAGGCGAAAATCC |
| | | TGTTTGATGGTGGTTAACGGCGGGATATAACATGAGCTGTCT |
| | | TCGGTATCGTCGTATCCCACTACCGAGATGTCCGCACCAACG |
| | | CGCAGCCCGGACTCGGTAATGGCGCGCATTGCGCCCAGCGCC |
| | | ATCTGATCGTTGGCAACCAGCATCGCAGTGGGAACGATGCCC |
| | | TCATTCAGCATTTGCATGGTTTGTTGAAAACCGGACATGGCA |
| | | CTCCAGTCGCCTTCCCGTTCCGCTATCGGCTGAATTTGATTG |
| | | CGAGTGAGATATTTATGCCAGCCAGCCAGACGCAGACGCGCC |
| | | GAGACAGAACTTAATGGGCCCGCTAACAGCGCGATTTGCTGG |
| | | TGACCCAATGCGACCAGATGCTCCACGCCCAGTCGCGTACCG |
| | | TCTTCATGGGAGAAAATAATACTGTTGATGGGTGTCTGGTCA |
| | | GAGACATCAAGAAATAACGCCGGAACATTAGTGCAGGCAGCT |
| | | TCCACAGCAATGGCATCCTGGTCATCCAGCGGATAGTTAATG |
| | | ATCAGCCCACTGACGCGTTGCGCGAGAAGATTGTGCACCGCC |
| | | GCTTTACAGGCTTCGACGCCGCTTCGTTCTACCATCGACACC |
| | | ACCACGCTGGCACCCAGTTGATCGGCGCGAGATTTAATCGCC |
| | | GCGACAATTTGCGACGGCGCGTGCAGGGCCAGACTGGAGGTG |
| | | GCAACGCCAATCAGCAACGACTGTTTGCCCGCCAGTTGTTGT |
| | | GCCACGCGGTTGGGAATGTAATTCAGCTCCGCCATCGCCGCT |
| | | TCCACTTTTTCCCGCGTTTTCGCAGAAACGTGGCTGGCCTGG |
| | | TTCACCACGCGGGAAACGGTCTGATAAGAGACACCGGCATAC |
| | | TCTGCGACATCGTATAACGTTACTGGTTTCACATTCACCACC |
| | | CTGAATTGACTCTCTTCCGGGCGCTATCATGCCATACCGCGA |
| | | AAGGTTTTGCGCCATTCGATGGTGTCCGGGATCTCGACGCTC |
| | | TCCCTTATGCGACTCCTGCATTAGGAAATTAATACGACTCAC |
| | | TATA |

Key:
Upper case nucleotides = DNA; Lowercase nucleotides = RNA; Underlined lowercase = 2′-O-methyl RNA; C3 = C3 spacer (propanediol modifier); * = phosphorothioate intemucleotide linkage; and +a, +c, +t, +g = LNA.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated crRNA comprising a length-modified and chemically modified form of formula (I):
5′-X—Z-3′ (I);
wherein X is a target-specific protospacer domain and Z is a tracrRNA-binding domain;
wherein the tracrRNA binding domain further comprises at least one chemically modified nucleotide;
and wherein the isolated crRNA is active in a Clustered Regularly Interspaced Short Palindromic Repeats (CRISPR)/CRISPR-associated protein endonuclease system.

2. The isolated crRNA of claim 1, wherein the protospacer domain consists of 17, 18, 19 or 20 nucleotides.

3. The isolated crRNA of claim 1, wherein the at least one chemically modified nucleotide is at or near the 3′ end.

4. The isolated crRNA of claim 1, wherein the at least one chemically modified nucleotide consists of a 2-O-Methyl modification, a phosphorothioate internucleotide linkage, a locked nucleic acid, or a combination thereof.

5. The isolated crRNA of claim 1, wherein the tracrRNA-binding domain is selected from the group consisting of SEQ ID No. 41, SEQ ID No. 42, SEQ ID No. 43 and SEQ ID No. 44.

6. A method of performing gene editing, comprising: contacting a candidate editing target site locus with an active CRISPR/Cas endonuclease system having a suitable crRNA;
wherein the crRNA has a tracrRNA binding domain; and
wherein the tracrRNA binding domain further comprises at least one chemically modified nucleotide.

7. The method of claim 6, wherein the tracrRNA binding domain is selected form the group consisting of SEQ ID No. 41, SEQ ID No. 42, SEQ ID No. 43 and SEQ ID No. 44.

8. A method of performing gene editing, comprising: contacting a candidate editing target site locus in bacteria with an active CRISPR/Cas endonuclease system having a suitable crRNA;
wherein the crRNA has a tracrRNA binding domain; and
wherein the tracrRNA binding domain further comprises at least one chemically modified nucleotide.

9. The method of claim 8, wherein the tracrRNA binding domain is SEQ ID No. 46.
